(19) United States
(12) Patent Application Publication
(10) Pub. No.: US 2026/0249288 A1
(43) Pub. Date: Aug. 27, 2026

(54) TOPOGRAPHIC MODULATION OF ENZYMATIC REACTION AFFORDS ULTRASENSITIVE COMPARTMENT-FREE DIGITAL IMMUNOASSAYS

(71) Applicant: University of Florida Research Foundation, Inc., Gainesville, FL (US)

(72) Inventors: Yong ZENG, Gainesville, FL (US); Yunjie WEN, Gainesville, FL (US)

(21) Appl. No.: 18/875,756

(22) PCT Filed: Jul. 20, 2023
(86) PCT No.: PCT/US2023/070542

§ 371 Date: Dec. 17, 2024

Related U.S. Application Data
(60) Provisional application No. 63/391,607, filed on Jul. 22, 2022.

(51) Int. Cl. B01L 3/00; G01N 33/543

(52) U.S. Cl. CPC B01L 3/50273; B01L 3/502738; G01N 33/54366; B01L 2300/0822; B01L 2300/0832; B01L 2300/12; B01L 2300/161; B01L 2400/0487; B01L 2400/0633

(57) ABSTRACT

In one aspect, the disclosure relates to a system comprising a plurality of microposts protruding from a membrane opposite a solid substrate, wherein the system can be pneumatically actuated to bring the microposts closer to the solid substrate. Also disclosed herein are methods of constructing the system, methods of performing digital bioassays using the disclosed system, and noninvasive methods for detecting diseases and for monitoring the progress of disease treatments using the disclosed system. This abstract is intended as a scanning tool for purposes of searching in the particular art and is not intended to be limiting of the present disclosure.

### CROSS-REFERENCE TO RELATED APPLICATIONS

This application claims the benefit of U.S. Provisional Application No. 63/391,607, filed on Jul. 22, 2022, which is incorporated herein by reference in its entirety.

### STATEMENT REGARDING FEDERALLY SPONSORED RESEARCH OR DEVELOPMENT

This invention was made with government support under Grant Number R33 CA214333, awarded by the National Institutes of Health. The government has certain rights in the invention.

### BACKGROUND

Confinement of molecules occurs ubiquitously in nature and fundamentally affects their properties and associated reactions. In living systems, confinements are considered instrumental in mediating biological processes, including stabilization, storage, transportation, interactions, and synthesis of biomolecules. Such confinement effects have been shown to result in enhanced kinetics, extraordinary selectivity, and precise control of chemical reactions. For instance, enzymes in cells that are surface-bound or compartmentalized regulate a sophisticated network of biochemical reactions in an efficient, timely, spatially defined, and physiologically favored manner. Inspirations from nature have drawn extensive interest in exploring artificial nanoconfinement systems that imitate biological conditions to deliver these appealing features. These synthetic systems promise to address current challenges in a broad range of fields, including catalysis, energy, biosensing, and pharmaceutics.

Prevalent confining strategies encompass volumetric encapsulation using molecular or physical compartmentalization and surface-/interface immobilization of molecules on solid supports. Recent remarkable advances in nanomaterials and nanotechnology provide a myriad of promising platforms, such as nanoparticles, nanochannels, 2D materials, and nanoporous structures, to develop synthetic confining systems. These nanoscale materials and devices offer unique properties, such as ultrahigh surface-to-volume ratio and the ability to manipulate the spatial distribution and/or orientation of enzymes, to control the thermodynamics and kinetics of confined reactions. It was observed that nanoconfinements result in the acceleration of biochemical reactions, improved catalytic activities of enzymes, favorable shift in reaction equilibrium of antibody-antigen binding, and enhancement or alteration of selectivity. Developing new systems that precisely modulate nanoconfinement effects is essential to elucidating the principles governing confinement-modulated reactivity, which will shed new insights in complex biological processes and promote their broad applications.

An increasingly growing application of nanoconfinements is to develop new biosensing platforms. Various forms of nanoconfinements have been explored for biosensing, including nanoporous materials (e.g., graphene, metal-organic frameworks, and nanogels), nanocapsules, and nanofabricated devices. These nanoscale systems affect the biosensing processes via many factors, including large surface area, increased local concentration of reactants, promoted mass transfer, and enhanced physical interactions and molecular binding between the target and sensing agents, leading to the improved analytical sensitivity, specificity, and speed. Relevant to this work, recent advances in digital bioassays clearly demonstrate the power of nanoconfinements to revolutionize biosensing for nucleic acids and proteins. Digital bioassays, such as droplet digital polymerase chain reaction (ddPCR) and digital enzyme-linked immunosorbent assay (dELISA), commonly involve stochastic encapsulation of individual target molecules into a large number of compartments of femtoliter (fL) to nanoliter (nL) volume in a “one-or-none” manner. Such small-volume confinements have been shown to immensely enhance the efficiency of the confined single-molecule enzymatic reactions, which enables digital quantification of the low-level target molecules with much improved sensitivity, specificity, and accuracy. A variety of strategies have been investigated for single-molecule compartmentalization, including the standard droplet microfluidics and microwell-based methods, in addition to less common methods, such as porous gel particles and surface wettability patterning. These methods require complex devices and operational processes for compartmentalization, such as sophisticated fabrication of fL microwell arrays and multi-phase microfluidics for droplet generation or microwell sealing. Another major factor that affects the sensitivity and precision of current dELISA arises from the low sampling efficiency which limits the number of detectable events, resulting in the large Poisson noise and measurement uncertainty for single-molecule counting at low concentration. New digitization technologies are desired to promote the development of next-generation digital biosensing and their broad use.

Despite advances in biosensing and nanoconfinement research, there is still a scarcity of methods that do not require construction of femtoliter to nanoliter scale compartments, that do not require large numbers of magnetic beads, and that have adequate sampling efficiency when used for enzyme assays and other enzyme reactions including, but not limited to, dELISA. These needs and other needs are satisfied by the present disclosure.

### SUMMARY

In accordance with the purpose(s) of the present disclosure, as embodied and broadly described herein, the disclosure, in one aspect, relates to a system comprising a plurality of microposts protruding from a membrane opposite a solid substrate, wherein the system can be pneumatically actuated to bring the microposts closer to the solid substrate. Also disclosed herein are methods of constructing the system, methods of performing digital bioassays using the disclosed system, and noninvasive methods for detecting diseases and for monitoring the progress of disease treatments using the disclosed system.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1Ai-1F show mechanistic studies of the disclosed microfluidic topographic modulation and intensification of enzymatic reaction (μTUNER) technology. (FIGS. 1Ai-1Aiv) Conceptual illustration of the μTUNER strategy that modulates and enhances surface enzymatic reactions. Surface enzymatic reactions in the enzyme-coated microreactor (FIG. 1Ai) can be conducted in the unperturbed mode (FIG. 1Aii) or in the modulation mode (FIG. 1Aiii). Alkaline phosphatase (ALP)/ELF-97 (FIG. 1Aiv) as the enzyme/substrate pair is investigated in this study. (FIG. 1B) Top, proposed enzyme kinetics model for ALP/ELF-97 reaction. Bottom, schematic of the species transport process underlying the μTUNER process. Four species are identified in the system: E, S, P_{(aq)}, and P(s). The diffusive transport of S and P_{(aq)} and the precipitation of P_{(aq)} are highlighted. The thickness of colored arrows represents the proceeding extent of a specific process. The thicker the arrow is, the more extent the process proceeds to. (FIG. 1C) Simulation results showing the time evolution of the surface concentration profile of P(s). Post diameter (d)=15 μm. Simulation rate constants: k₁=10⁻⁶ m/s, k₂=10⁻⁸ m/s, and kₚ=10³ s⁻¹. The sectional concentration profile at y=0 mm and the projection of the surface concentration profile are also displayed. Color contours indicate the concentration magnitude. (FIG. 1D) Simulation results showing the surface concentration profile of P(s) at t=120 s. d=40 μm. Simulation rate constants: k1=10⁻⁶ m/s, k₂=10⁻⁸ m/s, and kₚ=10³ s⁻¹. The sectional concentration profile at y=0 mm and the projection of the surface concentration profile are also displayed. Color contours indicate the concentration magnitude. (FIG. 1E) Simulation results showing the surface concentration profile of P₍ₛ₎ at t=120 s with k₁ increased to 10⁻⁵ m/s. d=15 μm. k₂=10⁻⁸ m/s. kₚ=10³ s⁻¹. The sectional concentration profile at y=0 mm and the projection of the surface concentration profile are also displayed. Color contours indicate the concentration magnitude. (FIG. 1F) Simulation results comparing the enhancement of P(s) concentration using different gap heights at y=0 mm and t=120 s. d=15 μm. Simulation rate constants: k₁=10⁻⁵ m/s, k₂=10⁻⁸ m/s, and kₚ=10³ s⁻¹.

FIGS. 2A-2E show characterization of the μTUNER chip. (FIG. 2A) Design of the μTUNER chip composed of a pneumatic control circuit and an array of four parallel microreactors patterned with the micropost arrays. (FIG. 2B) Digital photo of a μTUNER chip showing the microreactor array with the micropost structures. The diameter and spacing of the microposts are both 15 μm as shown in the magnified image. (FIG. 2C) Optical profilometry plot of the array of 15-μm microposts. The fabricated microposts show a conical frustum shape with a slightly reduced top diameter of 11.6±0.9 μm. (FIG. 2D) Snapshots of λ-DNA molecules confined by microposts of 80-μm diameter at pressing pressures of 0 kPa, 10 kPa, 20 kPa, and 40 kPa. (FIG. 2E) Scaled in-plane radius of gyration of λ-DNA (R∥/R∥_{,bulk}) determined at different pressing pressures. Reference plots of R∥/R∥_{,bulk} as a function of slit height reported from Tang et al.80 and Lin et al. are also included. Slit-like gap heights at different pressures are estimated to be: H_{0 kPa}≈1.3-2 μm, H_{10 kPa}≈270-600 nm, H_{20 kPa}≈100-190 nm, and H_{40 kPa}≈50-80 nm.

FIGS. 3A-3J show surface enzymatic reaction enhancement and signal patterning by μTUNER. (FIG. 3A) Comparisons of ALP/ELF-97 reaction conducted in the unperturbed mode (top) or the modulation mode (bottom). Post diameter (d)=post spacing (l)=15 μm. Scale bars, 100 μm. (FIG. 3B) 3D confocal fluorescence microscopy image showing the spatial distribution of ELF-97 alcohol precipitates inside the microreactor. d=l=15 μm. (FIG. 3C) Left, representative fluorescence microscopy images of ALP/ELF-97 reaction conducted using post array designs (d/l) of 20/20 μm (top) and 20/80 μm (bottom). Right, surface plots of the images showing the fluorescence intensity. Scale bars, 100 μm. (FIG. 3D) Left, fluorescence images of ALP/ELF-97 reaction conducted using post array designs (d/l) of 40/40 μm (top) and 160/160 μm (bottom). Right, surface plots of the images showing the fluorescence intensity. Scale bars, 100 μm. (FIG. 3E) Time-lapse plots of the fluorescence intensity at three designated locations (left) using 10-μm (middle) and 40-μm (right) posts. Error bars represent one S.D. (n=3). (FIG. 3F) Effects of reaction kinetics and mass transport on the nano-confined surface reaction. Left, fluorescence images of ALP/ELF-97 reaction using 2.5 mM ELF-97 and 20 kPa pressing pressure. Right, fluorescence images of ALP/ELF-97 reaction using 2.5 mM ELF-97 and 0 kPa pressing pressure. Surface plots of the images showing the fluorescence intensity are also displayed. d=I=15 μm. Scale bars, 100 μm. (FIG. 3G) Michaelis-Menten curves fitted from the apparent initial reaction rates measured at the three designated locations as a function of substrate concentration. Shadow areas indicate 95% confidence bands of the fitting curves. Error bars represent one S.D. (n=5). (FIG. 3H) Fluorescence image of the pattern of UF hallmarks and Florida Gators printed by contactless spatial modulation of the enzymatic production of ELF-97 alcohol precipitates. Scale bar, 100 μm. Inset, optical profilometry plot of the Florida Gators pattern in a positive stamp fabricated by photolithography. Color contours indicate the depth magnitude. (FIG. 3I) Top, principles of horseradish peroxide (HRP)/tyramide reaction and the deposition of dye-labeled tyramide substrate on a surface protein. Bottom, schematic of the μTUNER-modulated HRP/tyramide reaction. (FIG. 3J) Top, representative fluorescence microscopy image of HRP/tyramide reaction conducted using a mixture array of 10-μm and 40-μm posts. Scale bar, 100 μm. Bottom, fluorescence intensity profile of the image is displayed for the position indicated by the arrow.

FIGS. 4A-4E show μTUNER-based dELISA. (FIG. 4A) Schematics of the μTUNER-enabled dELISA. (FIG. 4B) Fluorescence images for detection of CD99 at variable concentrations. (FIG. 4C) Comparison of the ratios of fluorescent dots under the microposts and in the open channel area under different concentrations. (FIG. 4D) Calibration curves for quantifying CD99 by the μTUNER and conventional microfluidic chip. Inset: determination of LOD for measuring CD99 by the μTUNER digital immunoassay from 3 SDs of the backgrounds (dashed lines). (FIG. 4E) Calibration curves for quantifying NGFR, EZR, and ENO-2 by the μTUNER chip. Inset: determination of LODs for measuring each biomarker from 3 S.D.s of the backgrounds (dashed lines).

FIGS. 5A-5E show detection of protein markers in EWS cell line-derived sEVs with the μTUNER digital immunoassay. (FIG. 5A) Nanoparticle tracking analysis (NTA) measurements of the abundance and size distribution of EVs isolated from conditioned culture media of Hs919.T (normal control) and two EWS cell lines, CHLA-9 and CHLA-258. (FIG. 5B) Capillary Western analysis of the expression of CD81 and four EWS candidate markers (CD99, EZR, NGFR, and ENO-2) in Hs919.T, CHLA-9, and CHLA-258 cell-derived sEVs. (FIG. 5C) Quantitative digital detection of the four proteins in serially diluted Hs919.T, CHLA-9, and CHLA-258 sEVs. The dashed lines represent calibration curves obtained by least squares regression fitting. Error bars: one S.D. (n=3). (FIG. 5D) The expression levels of four sEV markers in Hs919.T, CHLA-9, and CHLA-258 sEVs measured by the μTUNER digital immunoassay. EV concentration used was 108 vesicles/mL for each cell line. EV protein concentrations were calculated from the measured digital counts using the calibration curves established in FIGS. 4A-4E. Error bars are one S.D. (n=3). Statistical comparisons between the control and each EWS cell line were performed by two-tailed Student's t-test. Significance level was set at p<0.05. (FIG. 5E) Heatmap obtained by non-supervised hierarchical clustering of the measured levels of four sEV protein markers that differentiates the three cell lines. Clustering analysis was performed with Ward linkage and Euclidean distance at the 95% confidence level.

FIGS. 6A-6I show clinical profiling of plasma EV biomarkers for EWS diagnosis with the μTUNER digital immunoassay chip. (FIG. 6A) Representative TEM images of sEVs isolated from an EWS plasma sample by UC. The scale bars in the low- and high-magnification images are 200 and 100 nm, respectively. (FIG. 6B) NTA measurements of UC-isolated plasma sEVs from a healthy donor (control), an adult EWS, and a pediatric EWS patient. (FIG. 6C) Quantification of the levels of four candidate markers (CD99, EZR, NGFR, and ENO-2) in plasma sEVs derived from the non-cancer controls (n=17) and the EWS patients (n=16). The EWS group consists of 5 adults and 11 pediatric patients. The protein levels were determined from the background-subtracted signals using the established calibration plots. Each sample was assayed in triplicate and the error bars indicate S.D. (n=3). (FIG. 6D) Superimposed scatter dot and violin plots of individual sEV protein markers and the SUM signature for detecting the EWS group from the control group. The SUM signature denotes the unweighted sum of four sEV markers. Boxes: mean±95% Cl; Whisker bars: S.D.; Dash lines: optimal cut-off values. Statistical two-group comparison was done using the non-parametric, two-tailed Mann-Whitney U-test (p<0.05). (FIG. 6E) ROC curves and AUC analysis of the individual and combined sEV protein biomarkers for EWS diagnosis. (FIG. 6F) Non-supervised hierarchical clustering of the measured levels of four sEV markers correctly classified the subjects into the control and EWS groups. Clustering analysis was performed with Ward linkage and Euclidean distance at the 95% confidence level. (FIGS. 6G-6I) Multi-group classification of individual subjects conducted using quadratic discriminant analysis of the four-marker panel was presented in (FIG. 6G) the biplot of the first two canonical variables with the vectors displaying the variance contribution of individual sEV markers to the discrimination among the groups, (FIG. 6H) the heatmap of post probabilities, and (FIG. 6I) the confusion matrix for quantitative assessment of classification performance. Dashed ellipses in (FIG. 6G) represent 95% confidence intervals for the means of predicted groups. All statistical analyses were performed at 95% confidence level.

FIGS. 7A-7E show a simulation model of the micropost-induced perturbation of surface enzymatic reaction. (FIG. 7A) 3D simulation model of a microreactor. The depth, width, and height of the microreactor are 3d, 3d, and H+h. The coordinate origin is at the center of the bottom boundary of the microreactor. The height and diameter of the post is h and d. The nanogap height is H, which is the distance between the bottom boundary of the post and the bottom boundary of the microreactor. (FIG. 7B) An exemplary 3D model with a 40 μm post and a 100 nm thick nanogap showing the gradient structured mesh with the smallest mesh size and the highest mesh density at the surface under the post to appropriately capture the behaviors of the surface reaction and mass transfer in the nanogap. (FIG. 7C) The cross-sectional plane at y=0 and (FIG. 7D) magnified diagram of the cross-sectional plane. The distance between the side boundaries of the post and microreactor is also d. (FIG. 7E) An exemplary 2D model with a 15 μm post and a 100 nm thick nanogap showing the gradient structured mesh used to appropriately capture the behaviors of the surface reaction and mass transfer in the nanogap.

FIGS. 8A-8B show nanogap confinement enhances the surface enzymatic reaction rate to reach the saturation level of P_{(aq)}. (FIG. 8A) Three selected locations on the reaction surface (z=0) to observe the effects of nanogap confinement on reaction rates. The coordinates of the three points are Location A (0, 0), Location B (−0.45d, 0), and Location C (−d, 0). The dashed line indicates the location of the micropost projected onto the reaction surface. (FIG. 8B) Simulation results of the time evolution of P_{(aq)} concentration at locations A, B, and C. Location A showed the highest reaction rate to reach the saturation level of P_{(aq)} (cₛₐₜ=10⁻⁵ M), followed by Location B. Location C had the slowest reaction rate. Simulation model geometry: d=15 μm and H=100 nm.

FIG. 9 shows the concentration profile of S on the cross-sectional plane (y=0) at t=1 s. Color contour indicates the concentration magnitude. The concentration gradient of S from the bulk space to the nanogap is significantly larger than that across the open bottom surface. Simulation model geometry: d=15 μm and H=100 nm.

FIGS. 10A-10C show multi-post systems yield consistent reaction modulation behavior compared with the single-post system. The geometries of three 2D simulation models with one (FIG. 10A), three (FIG. 10B), and five (FIG. 10C) microposts (top panel) and the simulated P₍ₛ₎ concentration profile on the reaction surface obtained at two reaction time points, t=1 s (middle) and t=120 s (bottom) using these models. The post diameter, post interval, and the distance between the outermost post and the microreactor side boundary are all set as d. The side boundaries of the microreactor slightly affected the quantitative results of the P₍ₛ₎ concentration, but both the multi-post and single-post systems yielded consistent behavior with the most enhanced reaction at the gap entrance and suppressed reaction at the center. Simulation model geometry: d=15 μm and H=100 nm. Simulation rate constants: k₁=10⁻⁵ m/s, k₂=10⁻⁷ m/s, and kₚ=10³ s⁻¹.

FIGS. 11A-11C show assessment of the nanogap confinement effects by tuning surface reaction kinetics. P₍ₛ₎ concentration profile on the reaction surface simulated with k1 set to (FIG. 11A) 10⁻⁵ m/s, (FIG. 11B) 10⁻⁶ m/s, and (FIG. 11C) 10⁻⁷ m/s. Each k₁ value was assessed with k₂ decreased from 10⁻⁷ m/s to 10⁻⁸ m/s and to 10⁻⁹ m/s. Simulation model geometry: d=40 μm and H=100 nm. kₚ is set as 1000 s⁻¹ for all the cases.

FIG. 12 shows time-lapse plots of the fluorescence intensity measured at the three designated locations using different ELF-97 concentrations. The steady-state assumption

$$( \frac{d [ ES ]}{dt} \approx 0 )$$

for Michaelis-Menten model holds validity given the linearity of the rate of formation of fluorescent precipitates with respect to the time window of the measurement (the first five time points). Measured fluorescence intensity were presented as mean±1 S.D. (n=5).

FIGS. 13A-13B show optimizations of detection antibody concentration and enzymatic reaction time for the μTUNER-based dELISA. (FIG. 13A) Optimizations of the detection antibody concentration for the four protein targets. 10 pg/mL standard protein and 100 μg/mL capture antibody were used for optimizations. The detection antibody concentrations were labeled in the graph. For the optimized concentration, ~1 μg/mL detection antibody was chosen. Measured S/N were presented as mean±1 S.D. (n=3). (FIG. 13B) Optimization of the enzymatic reaction time. 250 μM ELF-97 and 0.3 μg/mL ALP were used. The number of digital counts almost approached the plateau after 30 min, and 30 min was picked as the final reaction time.

FIG. 14 shows Nanoparticle Tracking Analysis (NTA) results of small EVs isolated from 33 plasma samples by ultracentrifugation. For each sample, 0.2 mL plasma was processed to yield 20 μL purified EVs in PBS, which was then diluted for NTA. Measured EV sizes were presented as its mean value.

FIG. 15 shows characterization of the UC-isolated sEVs from 33 plasma samples. The abundance (left) and mean size (right) were measured by the ZetaView® (Particle Metrix) NTA. The error bars in the dot plots indicate the mean and one S.E.M. P values were determined by two-tailed Mann-Whitney U test.

FIG. 16 shows assessment of the influence of residual free proteins in the sEV preparation. The dELISA detection of ENO-2 was assessed using the same UC-isolated plasma sEV sample with and without the lysis treatment. Error bars indicate one S.D. (n=3).

FIG. 17 shows a box-whiskers charts with overlapped data points for comparing the measured expression levels of individual sEV protein markers and the SUM signature across the control, adult EWS, and pediatric EWS groups. The middle lines and the box depict the mean±one standard error; and the Whisker range indicates the 95% confidence interval of the mean. Statistical difference was determined by the Kruskal-Wallis one-way ANOVA with post hoc Dunn's test for the pairwise comparisons. All statistical analyses were performed at 95% confidence level.

FIG. 18 shows evaluation of the diagnostic performance of the four-EV-marker panel for clinical classification of the control, adult EWS, and pediatric EWS groups. Classification of individual subjects was conducted using the quadratic discriminant analysis because the within-group covariance matrices were tested to be unequal. The predicted probabilities yielded by QDA were used to conduct ROC curves and AUC analysis. All statistical analyses were performed at 95% confidence level.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

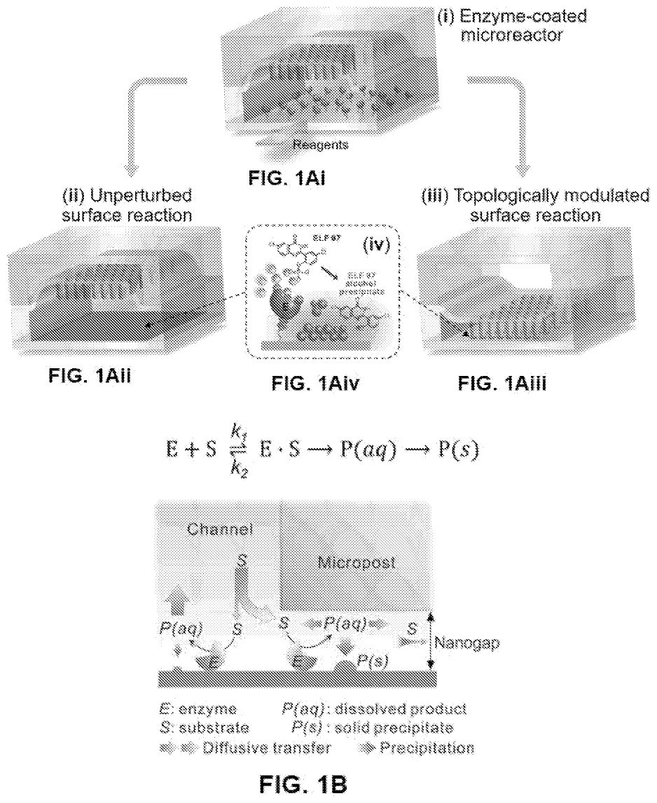

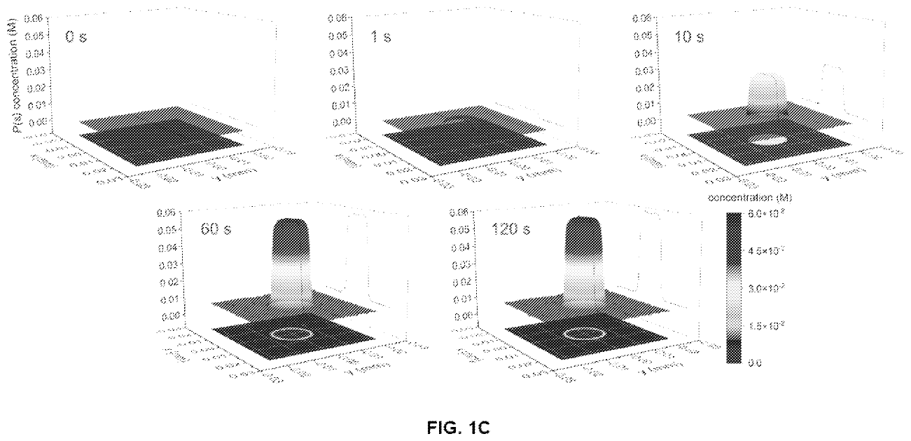

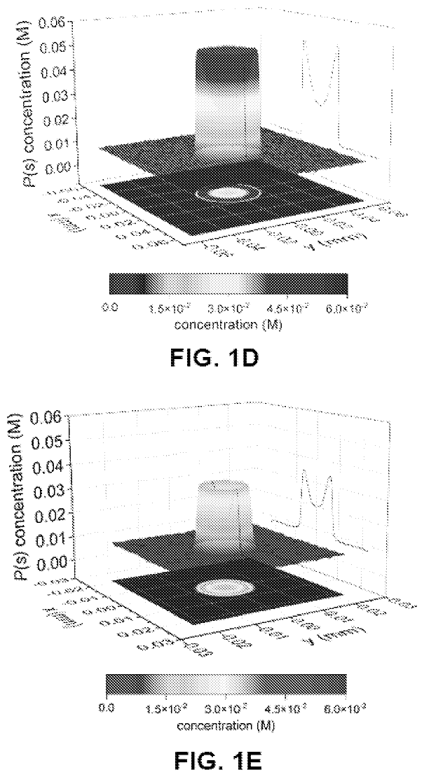

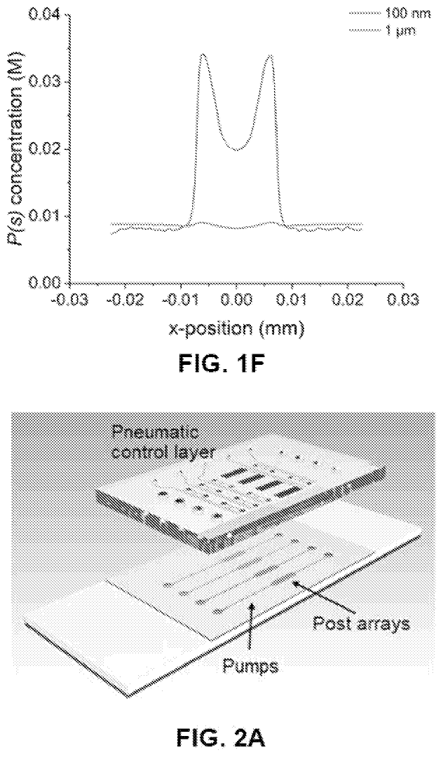

Herein is described a simple and robust nanoconfinement system that affords configurable mechanical modulation of surface enzymatic reaction termed microfluidic Topographic modUlation and iNtensification of Enzymatic Reaction (μTUNER). The μTUNER system is designed to pneumatically actuate a micropost array to create adjustable nanogaps between the microposts and the glass substrate (FIGS. 1Ai-1Aiv). Both numerical simulations and experimental investigations have been conducted to demonstrate the ability of the disclosed nanoconfinement approach to modulate the interplay between mass transfer and the kinetics of surface-immobilized enzyme reactions, resulting in spatial patterning of either enhanced or suppressed reactivity on the confined surface. In one aspect, the μTUNER presents a new strategy that repurposes a well-developed simple microdevice as an effective nanoconfinement system to enable configurable engineering and patterning of surface enzymatic reactions in a non-contact manner.

Also disclosed herein is a dELISA platform for quantitative single-molecule detection of low-level proteins. The disclosed method features several distinctions from the mainstream digital bioassay techniques. First, it negates the need for single-molecule compartmentalization using sophisticated partitioning and sealing devices, augmenting the adaptability of dELISA to general utilities. Moreover, the disclosed system adopts the surface-based ELISA format which further simplifies and expedites the analysis workflow by eliminating the multiple steps for conventional immunomagnetic capture and on-chip bead loading. In another aspect, the disclosed method also obviates the use of a large quantity of magnetic beads for immunocapture in the regime of Poisson distribution, further reducing the overall assay cost. Lastly, this surface-based approach can address the low sampling efficiency issue in standard dELISA to improve the analytical sensitivity and precision, because it permits continuous processing of large-volume sample to capture and accumulate rare target molecules and is not restricted by the pre-fixed number of reactions for digital counting.

As a proof-of-concept for potential clinical applications, the μTUNER-based dELISA device was adapted for liquid biopsy-based detection of Ewing sarcoma (EWS), the second most prevalent osseous malignancy in pediatric patients. EWS is an aggressive sarcoma with a high rate of metastasis present at time of diagnosis and thus associated with the most unfavorable prognosis of all the musculoskeletal tumors58. Pathological diagnosis and clinical staging require imaging (e.g., X-ray, computerized tomography, and/or positron emission tomography), tissue biopsies to evaluate for sheets of small, round blue cells which are often CD99+ by immunohistochemistry staining, and cytogenetic tests on the oncogenic chimeric fusions (most commonly EWSR1/FLI1) which is a hallmark of EWS. These standard approaches are often infeasible to repeat and associated with potential risks and high economic burden that can have a life-long impact on pediatric patients and their families. Moreover, major ethical challenges arise, including clinical equipoise, when considering an invasive biopsy procedure for children. Being often more aggressive and progressive compared to adult cancers, pediatric cancers, including EWS, could greatly benefit from repetitive, non-invasive tests for accurate diagnosis and treatment monitoring to improve therapeutic efficacy while minimizing toxic side effects. Liquid biopsy diagnostics is a rapidly arising paradigm to address these distinct challenges in the research, clinical trials, and patient care for pediatric cancer. Therefore, an extracellular vesicle (EV)-based clinical assay was developed that could be used for the diagnosis of EWS, detection of minimal residual disease, monitoring response to therapy, and early detection of disease relapse64. EVs, including exosomes, have been rapidly emerging as a promising new dimension of liquid biopsy for cancer diagnostics. Despite intensive studies of EVs in adult cancer, extremely limited efforts have been invested in childhood cancer to date.

With the disclosed μTUNER-based dELISA chip, four EWS-associated EV protein biomarkers identified in previous proteomic studies of EWS have been assessed. Highly sensitive and specific profiling of these markers (CD99, NGFR, ENO-2, and EZR) was demonstrated in EVs derived from cell lines and clinical plasma specimens. The expression levels of individual small EV (sEV) markers were found to afford appealing diagnostic performance for detecting the EWS patients from the control group; and the EV signature combining the four markers further improves the diagnostic power. With the aid of machine learning, this panel of four EV markers was also tested to demonstrate accurate classification of healthy donors, adult, and pediatric patients with an overall accuracy of 97%. Collectively, these results suggest that the μTUNER-enabled dELISA combined with the cancer specific EV biomarkers could provide a useful tool to improve clinical diagnosis, prognosis, and monitoring of EWS and other pediatric malignancies.

### Pneumatic-Actuated Micropost System

In one aspect, disclosed herein is a system having at least the following components:
(a) a first layer comprising solid substrate;
(b) a second layer comprising a plurality of microposts;
(c) a third layer; and
(d) a pneumatic actuator;
wherein the plurality of microposts protrude towards the first layer;
wherein the second layer is separated by an air gap from the third layer; and
wherein the pneumatic actuator can be activated to create a nanogap between the first layer and the plurality of microposts and to increase or decrease a height of the nanogap between the first layer and the plurality of microposts by increasing or decreasing a height of the air gap, thereby changing a position of the second layer.

In one aspect, the solid substrate can be a glass slide, such as, for example, a glass slide that has been subjected to a hydrophilic surface treatment. In another aspect, the solid substrate can be planar. Further in this aspect, a planar substrate does not include any wells, nanowells, fL capacity wells, or other constructed compartments for immobilization of antibodies or other reagents for the purpose of conducting digital bioassays. Without wishing to be bound by theory, contact or close proximity of the microposts to the substrate creates a compartmentalization effect without the need for specialized substrates.

In a further aspect, the second layer can be or include polydimethylsiloxane (PDMS), an addition cure silicone rubber comprising recycled polymers (e.g. EcoFlex™), polymethyl methacrylate (PMMA), or any combination thereof. In one aspect, the second layer can be from about 100 μm to about 300 μm thick, about 100 μm to about 200 μm thick, or can be about 150 μm thick. In still another aspect, the third layer can also be formed from polydimethylsiloxane (PDMS), an addition cure silicone rubber comprising recycled polymers, polymethyl methacrylate (PMMA), or any combination thereof. In an aspect, the third layer can be from about 4 mm to about 6 mm, thick, or can be about 5 mm thick.

In any of these aspects, the microposts can have a base diameter of from about 10 μm to about 200 μm, or from about 10 μm to about 160 μm, or from about 15 μm to about 80 μm. In another aspect, the microposts can have a conical frustum shape with a top diameter of from about 70% to about 100% of the base diameter, or from about 70% to about 90% of the base diameter, or from about 75% to about 80% of the base diameter.

In still another aspect, the microposts have a height of about 15 μm. In one aspect, the height of the nanogap can be adjusted between about 50 nm and about 2 μm or about 1.3 μm and about 1.75 μm. In some aspects, at very large pneumatic actuator pressures, the height of the nanogap can be about 0 nm.

In one aspect, the microposts are spaced from about 10 μm to about 200 μm apart, 10 μm to about 160 μm apart, from about 10 μm to about 100 μm apart, or from about 15 μm to about 80 μm apart, wherein spacing is measured between an edge of a first micropost and an edge of an adjacent micropost.

In some aspects, the system can further include at least one micropump, such as, for example, a 3-valve micropump or a syringe pump. In some aspects, the system configuration may need to be adjusted for use of a syringe pump. In another aspect, the system can include at least one microvalve such as, for example, a lifting gate microvalve.

In any of these aspects, the pneumatic actuator can be activated at a pressure of from about 0 kPa to about 40 kPa, about 10 kPa to about 30 kPa, or at about 20 kPa.

### Method for Performing a Digital Bioassay

In one aspect, disclosed herein is a method for performing a digital bioassay including at least the following steps:
(a) immobilizing a first reagent on the first layer of the disclosed system;
(b) flowing at least one additional reagent over the first layer; wherein a reaction between the at least one additional reagent and the first reagent produces a detectable signal if a target analyte is present; and
(c) reading the detectable signal;
wherein the at least one additional reagent is a liquid or is dissolved in a solvent; and
wherein the pneumatic actuator is activated to create the nanogap.

In a further aspect, the solvent can be water. In one aspect, activating the pneumatic actuator can trap a thin film of aqueous solution between the plurality of microposts and the first layer. In still another aspect, the thickness of the film of aqueous solution can be selected by activating the pneumatic actuator at a corresponding pressure.

In any of these aspects, step (b) of the method can be conducted using stop-flow pumping.

In another aspect, the digital bioassay can be a digital enzyme-linked immunosorbent assay (dELISA). Further in this aspect, the pneumatic actuator can be activated at a pressure of about 20 kPa when the assay is dELISA. In one aspect, the detectable signal can be a fluorescence signal.

### Digital ELISA Bioassay

In one aspect, the digital bioassay can be a dELISA assay and the first reagent can be one or more capture antibodies. In another aspect, the at least one additional reagent can be a test sample, wherein if the test sample contains the target analyte, the target analyte is captured by the one or more capture antibodies. In still another aspect, the at least one additional reagent further includes one or more detection antibodies for the target analyte. In yet another aspect, the at least one additional reagent further includes an enzyme that binds to the detection antibody, a reagent that produces a signal molecule upon reaction with the enzyme, or both.

In any of these aspects, the method further includes sequentially flowing the test sample, the one or more detection antibodies, the enzyme, and the reagent that produces a signal molecule upon reaction with the enzyme through the system in separate steps. In still another aspect, a wash fluid can then be flowed through the system to remove unbound target analyte, unbound detecting enzymes antibodies, unbound enzyme, and unbound reagent that produces a signal molecule upon reaction with the enzyme after each separate step.

In some aspects, the enzyme can be alkaline phosphatase, while the reagent that produces a signal molecule upon reaction with the enzyme can be 2-(5′-chloro-2-phosphoryloxyphenyl)-6-chloro-4(3H)-quinazolinone (ELF-97). Further in this aspect, the signal molecule can be a fluorescent alcohol, and the detectable signal can be a fluorescence signal. In a further aspect, the fluorescence signal can have an emission centered at about 530 nm.

### Method for Detecting a Disease

Also disclosed herein is a method for detecting a disease in a subject, the method including performing the disclosed method on a biological sample from the subject to detect at least one biomarker associated with the disease, wherein the at least one biomarker is the target analyte. In a further aspect, the subject can be a pediatric subject or an adult subject, while the biological sample can be a blood sample, a serum sample, a plasma sample, a urine sample, a cerebrospinal fluid (CSF) sample, bronchoalveolar lavage fluid, a saliva sample, or any combination thereof. In an aspect, the biological sample can include small extracellular vesicles (sEVs).

### Method for Monitoring the Progress of a Disease Treatment

Also disclosed herein is a method for monitoring the progress of treatment of a disease in a subject, the method including performing the disclosed method on a biological sample from the subject to detect at least one biomarker associated with the disease a first time and a second time, wherein the first time occurs before treatment or at an earlier time point in a course of treatment than the second time, wherein the at least one biomarker is the target analyte.

In a further aspect, a decrease in a level of the at least one biomarker indicates the treatment is succeeding, while in an alternative aspect, an increase in, or no change in, a level of the at least one biomarker indicates the treatment is not succeeding.

In a further aspect, the subject can be a pediatric subject or an adult subject, while the biological sample can be a blood sample, a serum sample, a plasma sample, a urine sample, a cerebrospinal fluid (CSF) sample, bronchoalveolar lavage fluid, a saliva sample, or any combination thereof.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a micropost,” “a substrate,” or “a biomarker,” include, but are not limited to, mixtures or combinations of two or more such microposts, substrates, or biomarkers, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be therapeutic in terms of a partial or complete cure of a disease, condition, symptom or adverse effect attributed to the disease, disorder, or condition. The term “treatment” as used herein can include any treatment of Ewing sarcoma in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered Aspects, which should not be confused with the claims.

Aspect 1. A system comprising:
(a) a first layer comprising a solid substrate;
(b) a second layer comprising a plurality of microposts;
(c) a third layer; and
(d) a pneumatic actuator;
wherein the plurality of microposts protrude towards the first layer;
wherein the second layer is separated by an air gap from the third layer; and
wherein the pneumatic actuator can be activated to create a nanogap between the first layer and the plurality of microposts and to increase or decrease a height of the nanogap between the first layer and the plurality of microposts by increasing or decreasing a height of the air gap, thereby changing a position of the second layer.

Aspect 2. The system of aspect 1, wherein the solid substrate comprises a glass slide.

Aspect 3. The system of aspect 2, wherein the glass slide has been subjected to a hydrophilic surface treatment.

Aspect 4. The system of any one of aspects 1-3, wherein the solid substrate is planar.

Aspect 5. The system of any one of aspects 1-4, wherein the second layer comprises polydimethylsiloxane (PDMS), an addition cure silicone rubber comprising recycled polymers, polymethyl methacrylate (PMMA), or any combination thereof.

Aspect 6. The system of aspect 5, wherein the second layer is made from PDMS.

Aspect 7. The system of aspect 6, wherein the second layer is from about 100 μm thick to about 300 μm thick.

Aspect 8. The system of aspect 7, wherein the second layer is about 150 μm thick.

Aspect 9. The system of any one of aspects 1-8, wherein the third layer comprises PDMS, an addition cure silicone rubber comprising recycled polymers, polymethyl methacrylate (PMMA), or any combination thereof.

Aspect 10. The system of aspect 9, wherein the third layer is made from. PDMS.

Aspect 11. The system of any one of aspects 1-10, wherein the third layer is from about 4 mm thick to about 6 mm thick.

Aspect 12. The system of aspect 11, wherein the third layer is about 5 mm thick.

Aspect 13. The system of any one of aspects 1-12, wherein the microposts have a base diameter of from about 10 μm to about 200 μm.

Aspect 14. The system of aspect 13, wherein the microposts comprise a conical frustum shape with a top diameter from about 70% to about 100% of the base diameter.

Aspect 15. The system of any one of aspects 1-14, wherein the microposts have a height of about 15 μm.

Aspect 16. The system of any one of aspects 1-15, wherein the microposts are spaced from about 10 μm to about 200 μm apart, wherein spacing is measured between an edge of a first micropost and an edge of an adjacent micropost.

Aspect 17. The system of any one of aspects 1-16 wherein the height of the nanogap can be adjusted between about 50 nm and about 2 μm.

Aspect 18. The system of any one of aspects 1-17, further comprising at least one micropump and at least one microvalve.

Aspect 19. The system of aspect 18, wherein the micropump comprises a 3-valve micropump or a syringe pump.

Aspect 20. The system of aspect 18 or 19, wherein the microvalve comprises a lifting gate microvalve.

Aspect 21. The system of any one of aspects 1-20, wherein the pneumatic actuator is activated at a pressure of from about 0 kPa to about 40 kPa.

Aspect 22. A method for performing a digital bioassay, the method comprising:
(a) immobilizing a first reagent on the first layer of the system of any one of aspects 1-21;
(b) flowing at least one additional reagent over the first layer; wherein a reaction between the at least one additional reagent and the first reagent produces a detectable signal if a target analyte is present; and
(c) reading the detectable signal;
wherein the at least one additional reagent is a liquid or is dissolved in a solvent; and
wherein the pneumatic actuator is activated to create the nanogap.

Aspect 23. The method of aspect 22, wherein the pneumatic actuator is activated at a pressure of about 20 kPa.

Aspect 24. The method of aspect 22 or 23, wherein the solvent is water.

Aspect 25. The method of any one of aspects 22-24, wherein activating the pneumatic actuator traps a thin film of aqueous solution between the plurality of microposts and the first layer.

Aspect 26. The method of aspect 25, wherein a thickness of the thin film of aqueous solution can be selected by activating the pneumatic actuator at a corresponding pressure.

Aspect 27. The method of any one of aspects 22-26, wherein step (b) is conducted using stop-flow pumping.

Aspect 28. The method of any one of aspects 22-27, wherein the digital bioassay comprises a digital enzyme-linked immunosorbent assay (dELISA).

Aspect 29. The method of aspect 28, wherein the digital bioassay comprises dELISA and the first reagent comprises one or more capture antibodies.

Aspect 30. The method of aspect 29, wherein the at least one additional reagent comprises a test sample, wherein if the test sample comprises the target analyte, the target analyte is captured by the one or more capture antibodies.

Aspect 31. The method of aspect 29 or 30, wherein the at least one additional reagent further comprises a detection antibody, wherein the detection antibody binds to the target analyte.

Aspect 32. The method of any one of aspects 29-31, wherein the at least one additional reagent further comprises an enzyme that binds to the detection antibody, a reagent that produces a signal molecule upon reaction with the enzyme, or both.

Aspect 33. The method of aspect 32, further comprising sequentially flowing the test sample, the detection antibody, the enzyme, and the reagent that produces a signal molecule upon reaction with the enzyme through the system in separate steps.

Aspect 34. The method of aspect 32 or 33, wherein a wash fluid is flowed through the system to remove unbound target analyte, unbound detecting antibody, unbound enzyme, and unbound reagent that produces a signal molecule upon reaction with the enzyme after each separate step.

Aspect 35. The method of aspect 33, wherein the enzyme comprises alkaline phosphatase.

Aspect 36. The method of any one of aspects 32-34, wherein the reagent that produces a signal molecule upon reaction with the enzyme comprises 2-(5′-chloro-2-phosphoryloxyphenyl)-6-chloro-4(3H)-quinazolinone (ELF-97).

Aspect 37. The method of aspect 36, wherein the signal molecule comprises a fluorescent alcohol.

Aspect 38. The method of any one of aspects 22-37, wherein the detectable signal comprises a fluorescence signal.

Aspect 39. The method of aspect 38, wherein the fluorescence signal has an emission centered at about 530 nm.

Aspect 40. A method for detecting a disease in a subject, the method comprising performing the method of any one of aspects 22-39 on a biological sample from the subject to detect at least one biomarker associated with the disease, wherein the at least one biomarker is the target analyte.

Aspect 41. The method of aspect 40, wherein the subject comprises a pediatric subject or an adult subject.

Aspect 42. The method of aspect 40 or 41, wherein the biological sample comprises a blood sample, a serum sample, plasma sample, a urine sample, a cerebrospinal fluid (CSF) sample, bronchoalveolar lavage fluid, a saliva sample, or any combination thereof.

Aspect 43. The method of aspect 42, wherein the biological sample is a plasma sample.

Aspect 44. The method of any one of aspects 40-43 wherein the biological sample comprises small extracellular vesicles (sEVs).

Aspect 45. A method for monitoring the progress of treatment of a disease, the method comprising performing the method of any one of aspects 40-44 on a biological sample from the subject to detect at least one biomarker associated with the disease at a first time and a second time, wherein the first time occurs before treatment or at an earlier time point in a course of treatment than the second time, wherein the at least one biomarker is the target analyte

Aspect 46. The method of aspect 45, wherein a decrease in a level of the at least one biomarker indicates the treatment is succeeding.

Aspect 47. The method of aspect 45, wherein an increase or no change in a level of the at least one biomarker indicates the treatment is not succeeding.

Aspect 48. The method of any one of aspects 45-47, wherein the subject comprises a pediatric subject or an adult subject.

Aspect 49. The method of any one of aspects 45-48, wherein the biological sample comprises a blood sample, a serum sample, plasma sample, a urine sample, a cerebrospinal fluid (CSF) sample, bronchoalveolar lavage fluid, a saliva sample, or any combination thereof.

Aspect 50. The method of aspect 49, wherein the biological sample comprises small extracellular vesicles (sEVs).

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Materials and Methods

### Reagents

Biotin-labeled bovine serum albumin (BSA) was purchased from Sigma-Aldrich. Carboxyethylsilanetriol (disodium salt, 25% in water), N-hydroxysuccinimide (NHS), 1-ethyl-3-(3-dimethylaminopropyl) carbodiimide (EDC) hydrochloride, Blocker™ BSA (10% in PBS), A DNA, YOYO™-1 iodide, dithiothreitol (DTT), ELF-97 (ex/em: 345/530), and RIPA lysis buffer were obtained from Thermo Fisher Scientific. Streptavidin conjugated alkaline phosphatase (ALP) was purchased from R&D Systems. Tyramide amplification kit with HRP streptavidin and CF®640R dye tyramide was purchased from Biotium. The detailed information of antibodies used in these studies was listed in Table 1. 1×PBS and 1×TE buffers were obtained from Thermo Fisher Scientific and Integrated DNA Technologies, respectively. All other solutions were prepared with deionized water (18.2 MO-cm; Thermo Fisher Scientific). ALP and ELF-97 were prepared in 1×PBS which contains 25 mM Tris (Thermo Fisher Scientific), 10 mM MgCl₂ (Sigma-Aldrich), and 1% BSA (Thermo Fisher Scientific) (ALP working buffer, pH 7.4). HRP and CF®640R dye tyramide were prepared in PBS working solution (PBSW, pH 7.4) containing 1% BSA and tyramide amplification buffer provided by the manufacturer, respectively. Standard NGFR, ENO-2, 0099, and Ezrin protein were prepared in PBS working solution (PBSW, pH 7.4) containing 1% BSA.

TABLE 1
Proteins and Antibodies Used

| Name | Vendor | Catalog No. |
|---|---|---|
| Purified anti-human CD99 | Biolegend | 317302 |
| Human CD99 Biotinylated Antibody | R&D systems | BAF3968 |
| CD99 (Human) Recombinant | Abnova | H00004267-P01 |
| Protein (P01) | | |
| Human Enolase 2/Neuron-specific | R&D systems | DY5169-05 |
| enolase Duoset | | |
| Human NGFR/TNFRSF 16 Duoset | R&D systems | DY367 |
| Ezrin Antibody [Biotin] | Novus Biologicals | NBP2-52977B |
| Ezrin Antibody (6F1C3)-BSA Free | Novus Biologicals | NBP2-52464 |
| Human EZR/Ezrin Protein | Lifespan | LS-G55738 |
| (Recombinant) | Biosciences | |

### Numerical Simulation

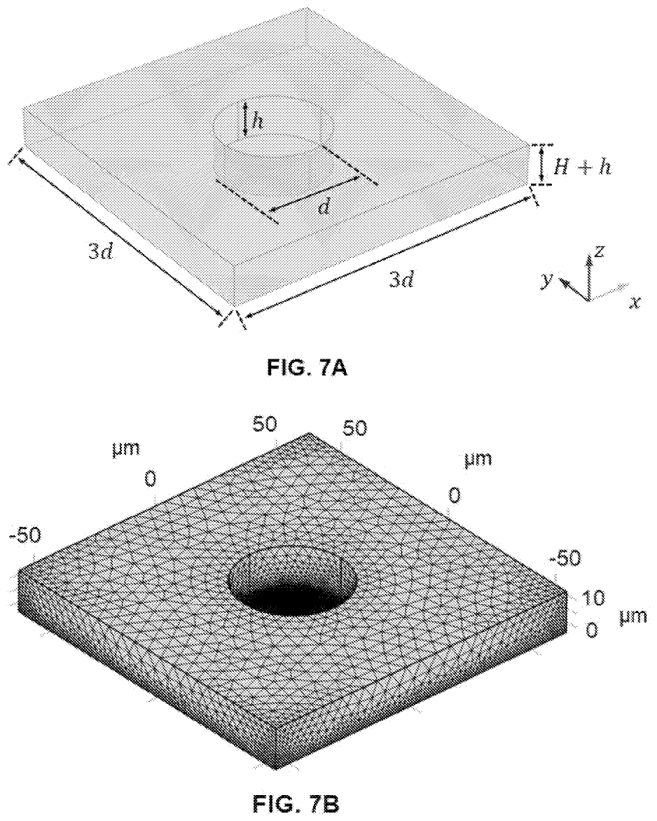

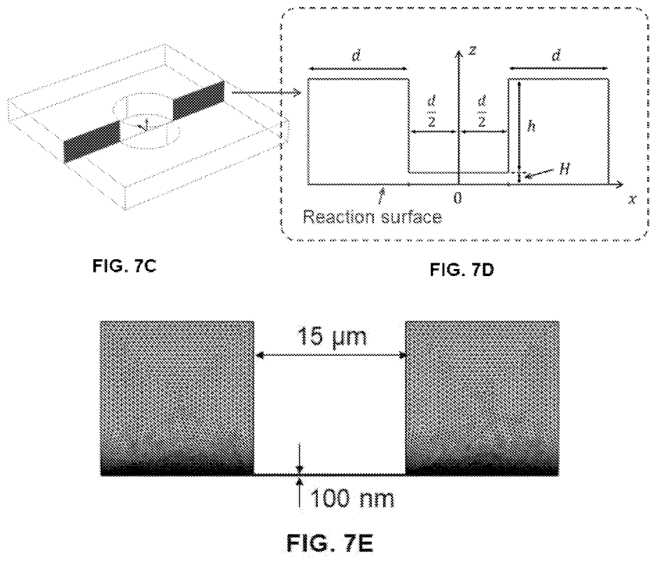

A computational species transport simulation was conducted using COMSOL Multiphysics to solve diffusion-reaction equations coupled with surface reactions through a finite-element approach. A simplified 3D geometry containing a single post that has identical dimensions to the experimental setups was used to model the micropost-induced perturbation of surface ALP/ELF-97 reaction (FIGS. 7A-7E). Diffusion coefficients of ELF-97 and dissolved ELF-97 alcohol molecules in PBS were estimated using Wilke-Chang correlation equations and are summarized in Table 2. A total of 53,850 and 339,867 elements were used for 3D 15-μm and 40-μm post design, respectively. A total of 23,571 and 65,143 elements were used for 2D 15-μm and 40-μm post design, respectively. Simulation equations and parameters can be found in Example 4.

TABLE 2
Simulation Constants

| Notation | Description | Value | |
|---|---|---|---|
| D_{A} | Diffusion coefficient of ELF-97^{a} | 6.3 × 10⁻¹⁰ m²/s | |
| D_{P1} | Diffusion coefficient of dissolved | 7.0 × 10⁻¹⁰ m²/s | |
| | ELF-97 alcohol^{a} | | |
| D_{P2} | Diffusion coefficient of precipitated | 0 | m²/s |
| | ELF-97 alcohol^{b} | | |
| Cₛₐₜ | Saturate concentration of dissolved | 10⁻⁵M | |
| | ELF-97 alcohol | | |
| C_{A}_0 | Initial concentration of ELF-97 | 500 | μM |

^{a}Diffusion coefficients of ELF-97 and dissolved ELF-97 alcohol molecules in PBS were estimated using Wilke-Chang correlation equations.
^{b}The precipitated product is assumed to be stationary.

### Microfabrication of Polydimethylsiloxane (PDMS) Chips

Two-layer PDMS chips were fabricated by multi-layer soft lithography according to an established protocol. Briefly, silicon wafers were cleaned with piranha solution and spin-coated with SU-8 photoresist (MicroChem). For the mold of fluidic layer, 15-μm thick SU-8 2010 was spin-coated. For the molds of pneumatic layer and surface patterning chip, 50-μm and 30-μm thick SU-8 2025 were spin-coated, respectively. The SU-8 microstructures were fabricated onto the wafers from the photomasks, following the protocols recommended by the manufacturer. Prior to use, the SU-8 molds were treated with trichloro(1H,1H,2H,2H-perfluorooctyl) silane under vacuum overnight. To fabricate the pneumatic layer, 35 g mixture of PDMS base and curing agent at a 10:1 ratio was poured on the mold and cured in the oven at 70° C. for 4 h. The PDMS slabs were peeled off from the mold, cut, and punched to make pneumatic connection holes. Meanwhile, the fluidic layer was prepared by spin-coating the mold with 5 g mixture of PDMS base and curing agent at a ratio of 10:1 at 500 rpm for 30 s, followed by 700 rpm for 30 s. It was then cured in the oven at 70° C. for 4 h. To assemble the pneumatic layer and fluidic layer, they were treated by UV-Ozone for 5 min and manually aligned together under a stereomicroscope and permanently bonded by baking in the oven at 70° C. overnight. The two-layer PDMS slabs were then peeled off from the mold and reservoirs were punched.

Measurement of Nanogap Heights with Fluorescence Imaging

The heights (H) of the slit-like gap at different actuation pressures were estimated via the fluorescence imaging of the conformational changes of individual λ-DNA molecules confined in the gap. λ-DNAs were firstly stained with YOYO-1 at a ratio of dye to base pair of 1:6 in 1×TE buffer (pH 8.0) containing 30 mM DTT at room temperature for 30 min. The λ-DNA solution was then 1:20 diluted in 1×TE buffer (pH 8.0). Microposts of 80 μm in diameter were chosen for the estimation of gap height. The μTUNER chip was firstly blocked with 5% BSA for 1.5 h, followed by washing with PBST, ddH₂O, and 1×TE buffer (pH 8.0) sequentially. The diluted A DNA solution was pumped in quickly to fill the chamber. The posts were then pressed down at different pressures (0 kPa, 10 kPa, 20 kPa, and 40 kPa). Valves on both sides were closed after pressing down the posts and the system was let stay for 5 min before imaging. The λ-DNA solution was re-pumped into the chamber each time for measurement at a new pressure. Imaging was performed using Zeiss Axio A1 fluorescence microscope with a 40× objective. The size of confined λ-DNA molecules was estimated by fitting them to homogeneous ellipses using ImageJ (NIH, http://rsbweb.nih.gov/ij/). Information of the radii of the fitting ellipse was obtained to calculate the average in-plane radius of gyration for λ-DNA molecules floating in the microchannel (R_{∥,bulk}) and confined under the posts (R_{∥}). Both R_{∥} and R_{∥,bulk} are given by

$$\sqrt{\frac{{R}_{M}^{2} + {R}_{m}^{2}}{2} ,}$$

with R_{M} and Rₘ the radii of the ellipse along major and minor axes, respectively. H is then extracted by comparing the scaled in-plane radius of gyration of λ-DNA (R_{∥}/R_{∥, bulk}) determined at different pressures and the reference plots of R_{∥}/R_{∥,bulk} as a function of slit height reported from two independent studies.

### Modulation of Surface Enzymatic Reaction by μTUNER

The μTUNER chip was surface functionalized via EDC/NHS reaction for protein/antibody conjugation. Briefly, the glass slide was pre-cleaned by piranha solution and treated with carboxyethylsilanetriol for 4 h. The glass slide was then washed with ddH₂O and treated with EDC/NHS solution (2.3 mg/mL NHS and 2 mg/mL EDC) for 1 h. After washing with ddH₂O, a patterning chip was assembled onto the glass slide and the solution of capture antibody/protein was flowed through the chip to coat the glass surface for 1 h at room temperature. The chip was then stored at 4° C. before the experiments. After removing the patterning chip, the surface-modified glass slide was dried by N₂. The two-layer PDMS flow-channel chip was treated by UV-Ozone for 5 min and was aligned and assembled onto the glass slide to construct the complete μTUNER chip. 500 μg/mL biotinylated BSA was used as the capture protein to coat the surface. The micropost array was lifted by vacuum to allow the reagents to flow through in each step. Solutions were pneumatically pumped through the channel in a “stop-flow” manner. The μTUNER chips were first blocked with 5% BSA for 1.5 h. Streptavidin-conjugated ALP was prepared by 1:500 dilution in ALP working buffer. 10 μL of diluted ALP (1.2 μg/mL) was then pumped through the channel and reacted for 0.5 h. After washing away unbounded enzymes with 30 μL PBST, 5 μL 500 μM ELF-97 in ALP working buffer was quickly pumped into the chamber in 20 s. After the chamber was filled with ELF-97, the micropost array was pressed down at 20 kPa, followed by closing the flanking valves to stop the fluid flow. The reaction was performed for 0.5 h and then fluorescence images were taken using a Nikon Eclipse Ti2 inverted fluorescence microscope with a 20× objective.

Microposts of 40 and 10 μm in diameter were used to compare the reaction kinetics under different micropost sizes by monitoring the real-time fluorescence signals. After pressing down the microposts at 20 kPa (t=0 s), fluorescence images were obtained at t=20 s, 40 s, 2 min, 4 min, 5 min, 7 min, and 10 min. Background subtracted fluorescence intensity was measured at the three designated surface locations: (A) the center of a post, (B) the inner point that is 2 μm from the post edge, and (C) the middle point between two adjacent posts to make the time-lapsed plots using ImageJ.

### Enzyme Kinetics Study Using the Michaelis-Menten Model

For experiments of enzyme kinetics study, a micropost array of 40 μm in diameter was used. ELF-97 of different concentrations was evaluated (500 μM, 750 μM, 1 mM, 1.25 mM, 1.5 mM, 2 mM, and 3 mM) with 1.2 μg/mL ALP. After the chamber is quickly filled with ELF-97, the micropost array was pressed down at 20 kPa. The moment when the micropost array was fully pressed down was manually picked as time zero point. The fluorescence images were taken every 2 s for 3 min followed by every 20 s for 7 min without moving the chip or camera view. Digital images were processed using ImageJ to measure the fluorescence intensity at the above-mentioned three surface locations. Five microposts were picked randomly to obtain the average fluorescence intensity. After obtaining the time-lapse plots, the apparent initial rate

$${v}_{0}^{app}$$

was calculated by linear fitting the first five points for each substrate concentration.

$${v}_{0}^{app}$$

was then plotted against the substate concentration to fit into the Michaelis-Menten model and the apparent Michaelis-Menten parameters,

$${K}_{M}^{app} and {V}_{max}^{app} ,$$

were obtained from the fitting curves.

### 3D Confocal Fluorescence Imaging

Confocal images were taken using a Nikon A1R MP Confocal/Multiphoton/STORM Microscope equipped with 405, 445, 488, 514, 561, and 647 nm solid-state lasers. A 60× long working distance oil objective was used. The laser intensity was 20% and the exposure time was 100 ms. Image stacks were taken at 0.5-μm interval along the z-axis ranging from the bottom of the glass substrate to the top of the pillar. The obtained image stacks were fitted into 3D view photography.

### Tyramide Signal Amplification (TSA) Reaction Modulated by μTUNER

A micropost mixture array of 40 and 10 μm in diameter was used. 10 μL of 2 μg/mL streptavidin conjugated HRP was injected and reacted for 1 h following blocking with 5% BSA for 1.5 h. After washing with 30 μL PBST, 5 μL 1×CF®640R dye tyramide was quickly pumped into the chamber in 20 s and the micropost array was pressed down at 20 kPa after the chamber was filled with the dye tyramides. The reaction went for 0.5 h at room temperature. The micropost array was then lifted up and 30 μL PBST was used to wash away the remaining dye tyramides. The micropost array was pressed down again at 20 kPa for fluorescence imaging.

### μTUNER Digital Immunoassay

The μTUNER chip was adapted to perform the on-chip sandwich ELISA to detect protein biomarkers in exosomes. Microposts of 15 μm in diameter were used for all measurements. To calibrate the μTUNER chip for quantitative detection of protein biomarkers, capture antibodies (100 μg/mL) of the four protein targets (CD99, NGFR, ENO-2, and EZR) were firstly immobilized on the glass slide using a patterning chip, as described above. The chip was blocked with 5% BSA for 1 h. Then, 10 μL of standard proteins of different concentrations (1 μg/mL to 5 ng/mL) were flowed through the channel to react for 2 h. After washing with 30 μL PBST, 10 μL of biotinylated detection antibodies were flowed through the channel to react for 1 h. After washing away the unbound antibodies with 30 μL PBST, 10 μL 0.6 μg/mL streptavidin conjugated ALP was flowed through the channel to label the detection antibody within 0.5 h. Finally, following another round washing with PBST, 5 μL 500 μM ELF-97 was quickly pumped into the channel in 20 s and the microposts were then pressed down at 20 kPa. ELF-97 was incubated with enzymes for 0.5 h before fluorescence imaging. Fluorescence images were taken using a Nikon Eclipse Ti2 inverted fluorescence microscope with a 20× objective and analyzed with ImageJ to measure the signal counts and plot the calibration curves for each protein target. To compare the ratios of fluorescent dots under the microposts and in the open channel area, the fluorescence image was aligned with the bright field image at the same location to count the dots.

### Cell Culture and EV Isolation

Procedures for cell culture and the collection of EV-contained conditioned serum-free medium (SFM) were as previously described. Briefly, the Hs919.T cell line, derived from benign osteoid osteoma, was obtained from the American Type Culture Collection. CHLA-258, derived from a primitive neuroectodermal tumor (PNET) patient prior to treatment and CHLA-9, derived from a PNET patient who relapsed after chemotherapy, were obtained from the Children's Oncology Group (COG). All cell lines were cultured at 37° C. under a 5% humidified CO₂ atmosphere. CHLA-258 and CHLA-9 were maintained in Iscoves modified Dulbecco's medium (IMDM), supplemented with L-glutamine (3 mM), insulin, transferrin (5 mg/mL each), selenium (5 ng/mL), and 20% heat-inactivated EV-free FBS (whole medium). Hs919.T was maintained in DMEM with high glucose and L-glutamine, supplemented with 20% heat-inactivated EV-free FBS (whole medium). Cell lines were cultured with 10% penicillin streptomycin to prevent bacterial growth/contamination. The three cell lines were cultured in T175 cm² flasks until cellular sub-confluency of ~70%.

Conditioned media was collected and centrifuged at 2,500 rpms for 5 min to remove cellular debris. The supernatant was then centrifuged at 4° C. for 45 min at 10,000×g to remove microvesicles and then at 100,000×g for 2 h to enrich exosomes. The pellets were then washed once by resuspending in 10 mL of PBS and ultracentrifugated at 4° C. for 60 min at 110,000×g. After aspiration of the supernatant, EV pellets were resuspended in 100 μL PBS, aliquoted and stored at −80° C.

### Nanoparticle Tracking Analysis

Particle number and size distribution of EVs from cell lines and plasma samples were determined by nanoparticle tracking analysis (NTA) using ZetaView® (Particle Metrix). Samples were diluted in PBS to an acceptable concentration recommended by the manufacturer.

Detection of Protein Markers in Cell Line-Derived sEVs

Cell line-derived sEVs were firstly lysed with RIPA buffer for 30 min (v/v=1:2). The lysates were then serially diluted by 1% BSA. The diluted lysates were then analyzed using the μTUNER chip following the digital immunoassay procedure as described above.

### Capillary Western Blot (Wes) Analysis

Capillary Western analyses were performed using the ProteinSimple Wes System. 0.4 μg/μl of sEV samples from CHLA-9, CHLA-258 and Hs919.T cell lines were diluted with 0.1× Sample Buffer. Then 4 parts of diluted sample were combined with 1 part 5× Fluorescent Master Mix (containing 5× sample buffer, 5× fluorescent standard, and 200 mM DTT) and heated at 95° C. for 5 min. The Fluorescent Master Mix contains three fluorescent proteins that act as a ‘ruler’ to normalize the distance for each capillary because the molecular weight ladder is only on the first capillary and each capillary is independent. After this denaturation step, the prepared samples, blocking reagent, primary antibodies (1:50 dilution for CD81 (Proteintech 66866-1-lg), CD99 (Novus Biologicals NBP2-67019), Ezrin (R&D systems MAB72391), NGFR (Cell signaling D8A8) and Enolase-2 (R&D systems MAB51691), HRP-conjugated secondary antibodies and chemiluminescent substrate were dispensed into designated wells in an assay plate. A biotinylated ladder provided molecular weight standards for each assay. After plate loading, the separation electrophoresis and immunodetection steps take place in the fully automated capillary system.

EV Isolation from Plasma Samples

Patient plasma samples (200 μL each) were first centrifuged at 4° C. at 2,000×g for 10 min to remove large cell debris followed by 10,000×g for 45 minutes to remove large vesicles. The supernatant was then collected to pellet EVs at 4° C. for 120 min at 100,000×g. After aspiration of the supernatant, EV pellets were resuspended in 20 μL PBS and stored in low-retention tubes at −80° C.

### Transmission Electron Microscopy (TEM)

EVs were examined by transmission electron microscopy negative stain at UF ICBR Electron Microscopy, RRID: SCR_019146. Glow discharged, 400 mesh carbon coated formvar copper grid was floated onto 5 μL of aliquot exosome suspension (1:5 dilution in 0.1 M PBS) for 5 minutes and water washed. Excess solution was drawn off with filter paper, and grid was floated onto 1% aqueous uranyl acetate for 30 seconds. Stain was removed with filter paper, air dried, and examined with a FEI Tecnai G2 Spirit Twin TEM (FEI Corp., Hillsboro, OR) operated at 120 kV. Digital images were acquired with Gatan UltraScan 2k×2k camera and Digital Micrograph software (Gatan Inc., Pleasanton, CA).

### Clinical EV Marker Analysis Using μTUNER Chips

The translational aspect of this study has been approved by the appropriate institutional research ethics committee. As part of a collaboration between Children's Mercy (CM) in Kansas City, MO, and the Biospecimen Repository Core Facility (BRCF) at the KU Medical Center (HSC #5929, Director, A. Godwin), a pediatric sarcoma protocol (CMH IRB #13010015, Glenson Samuel, MD) has been established to obtain tumor and blood specimens from pediatric sarcoma patients undergoing treatment at CM. Blood samples from aged/race matched cancer-free (healthy) individuals were obtained once the volunteer (or legal guardian) provided written, informed consent in accordance with the HSC 5929 umbrella IRB approved protocol. All blood samples, both from CM and KUMC/KUCC, were processed and banked in the Pediatric Sarcoma Biobank within the BRCF. De-identified patient specimens, cancer-free controls, and their accompanying clinical data were handled in an anonymous (coded) fashion. Informed consent was obtained to publish research results.

Archival clinical plasma samples collected from an age and gender matched cohort of patients who represent most of this disease population were obtained from BCRF: non-cancer controls (n=17) and EWS patients (n=16) who consist of 11 pediatric (≤18 years of age) and 5 adult patients (19 years old and above), as listed in Table 3. Plasma sEVs were isolated by UC and subjected to quality check using different standard methods, as described above. For μTUNER-based dELISA analysis, UC-isolated EVs were firstly lysed by RIPA buffer (v/v=1:2) for 30 min. The lysates were then 20 times diluted with 1% BSA. 10 μL diluted lysates were then analyzed following the μTUNER dELISA procedure as detailed above.

TABLE 3
Clinical Plasma Sample Information

| ID in this | | | | |
|---|---|---|---|---|
| Document | Original ID | Status | Age | Gender |
| 1 | 030183 | Healthy | 39 | M |
| 2 | 500098 | Healthy | 20 | M |
| 3 | 035967 | Healthy | 38 | M |
| 4 | 035151 | Healthy | 29 | M |
| 5 | 039445 | Healthy | 21 | M |
| 6 | 500155 | Healthy | 30 | M |
| 7 | 035385 | Healthy | 20 | F |
| 8 | 028385 | Healthy | 27 | F |
| 9 | 5929-601028 | Healthy | 27 | M |
| 10 | 500169 | Healthy | 23 | F |
| 11 | 5929-600697 | Healthy | 27 | M |
| 12 | 500309 | Healthy | 24 | F |
| 13 | 033500 | Healthy | 18 | F |
| 14 | 034831 | Healthy | 18 | F |
| 15 | 039173 | Healthy | 18 | F |
| 16 | 500353 | Healthy | 25 | M |
| 17 | 035473 | Osteosarcoma | 20 | F |
| 18 | 038716 | Ewings | 20 | M |
| 19 | 018414 | Ewings | 37 | M |
| 20 | 026048 | Ewings | 33 | M |
| 21 | 009588 | Ewings | 19 | M |
| 22 | 601857 | Ewings | 20 | M |
| 23 | 030292 | Ewings | 15 | M |
| 24 | 18563-pt16 | Ewings | 12 | F |
| 25 | 18561-pt18 | Ewings | 16 | F |
| 26 | 18633-pt23 | Ewings | 16 | F |
| 27 | 18652-pt25 | Ewings | 17 | M |
| 28 | 28171-pt32 | Ewings | 17 | F/M |
| | | | | (transitioned |
| | | | | to male) |
| 29 | 29144-pt36 | Ewings | 1 | F |
| 30 | 31763-pt38 | Ewings | 13 | F |
| 31 | 33629-pt40 | Ewings | 6 | F |
| 32 | 38699-pt42 | Ewings | 17 | F |
| 33 | 38706-pt43 | Ewings | 12 | F |

### Statistical Analysis

Mean, standard deviation (S.D.), standard error of the mean (S.E.M.), and limit of detection (LOD) were calculated with standard formulas. For cell line analysis, two-tailed Student's t-test was performed for two-group comparison with a significance level of P<0.05. For the analysis of human specimen, two-group difference was assessed using non-parametric, two-tailed Mann-Whitney U-test and multi-group comparisons were conducted using Kruskal-Wallis one-way ANOVA with post hoc Dunn's test. Non-supervised hierarchical clustering analysis was performed with Ward linkage and Euclidean distance to generate a heat map with the dendrogram. ROC analyses were performed to determine the AUC values for individual biomarkers and the SUM signature. Machine learning analysis of the marker expression levels for clinical classification of individual subjects was conducted using the discriminant analysis for which the quadratic mode was chosen as the within-group covariance matrices were tested to be unequal. The predicted probabilities yielded were then used to conduct ROC curves and AUC analysis to evaluate the diagnostic performance of the EV marker panel for clinical classification. All statistical analyses were conducted at a 95% confidence level using Excel 2018, OriginPro 2019, JMP Pro 16, and GraphPad Prism 8.

### Example 2: Results

The μTUNER (microfluidic Topographic modUlation and iNtensification of Enzymatic Reaction) approach was inspired by a previous observation that a thin film of aqueous solution will be trapped by pneumatically pressing a PDMS microstructure onto a hydrophilic glass surface and the film thickness can be tuned by varying the actuation pressure. It is hypothesized herein that this phenomenon can be harvested to create configurable topographic confinements to spatially modulate and enhance enzymatic reactions on a planar surface. To test this hypothesis, a model system was investigated in which a pneumatically actuatable microreactor was used to perturb the enzymatic activity of alkaline phosphatase (ALP) immobilized on the substrate surface, as conceptually illustrated in FIGS. 1Ai-1Aiv. The device has a three-layer PDMS/glass construct in which the middle PDMS membrane is patterned by a micropost array with the same height as the flow channel and the glass surface of the reaction chamber is coated uniformly by ALP protein. The micropost array can be lifted by vacuum to quickly fill the microreactor with a solution of ALP substrate (FIG. 1Ai). For comparison, surface enzymatic reaction can be performed in an unperturbed mode with the post array held up (FIG. 1Aii) or in the modulation mode with the post array pressed down at variable pressures (FIG. 1Aiii). In both cases, the fluid flow in the microreactor is stopped to prevent hydrodynamic disturbance of the spatial distribution of the enzymatic reaction products. For this proof-of-principle study, a soluble, nonfluorescent substrate, ELF-97, was chosen, which can be hydrolyzed by ALP into insoluble, fluorescent ELF-97 alcohol that precipitates out (FIG. 1Aiv). This ALP/ELF-97 reaction provides a well-poised model for this study because its precipitate product is 1) tightly localized to the site of enzymatic activity for activity detection with superior spatial resolution, and 2) photostable and strongly fluorescent for reliable and sensitive signal detection. These unique characteristics permit convenient visualization of the micropost-modulated enzymatic activity landscape with good sensitivity and resolution using standard fluorescence microscopy imaging.

To facilitate the mechanistic study of the μTUNER process, numerical simulations of the micropost-induced perturbation of surface ALP/ELF-97 reaction were first conducted using a simplified enzyme kinetic model (see Examples 1 and 4 for the simulation details). This model couples a classic enzyme kinetics equation with a step of product precipitation which is assumed to be irreversible owing to the very low solubility and fast precipitation of ELF-97 alcohol (FIG. 1B, top). In the present case, the enzyme (E) is uniformly distributed on the bottom surface of the microreactor. The substrate (S) in the microchannel diffuses to the surface to react with the enzyme, producing the dissolved product (P(aq)) near the surface that will diffuse into the bulk solution (FIG. 1B, bottom). If the local concentration of P(aq) accumulates to reach the saturation level, it will precipitate to form the solid product, P(s). Submicron-scale confinements have been shown to enhance surface-bound enzyme reactions and affinity binding. Thus, it is hypothesized that compared to the open channel region, the surface enzymatic reaction confined under a micropost can be enhanced, creating a stronger concentration gradient of S to drive its preferential diffusive transport toward the confined area to sustain the fast reaction. This effect accelerates the production of P(aq) within the nanogap to reach the saturation level and precipitate out. The precipitating process will be further expedited in the nanogap as the micropost restricts the vertical diffusion of P(aq) into the bulk to boost its local concentration near the surface. Because the micropost also restricts the mass transport of S from the bulk into the nanogap, the overall confinement effect is determined by the dynamic competition between the surface reaction and the replenishment of S via lateral diffusion along the radius of the micropost. At the sites near the micropost edge with low spatial impedance on diffusion, the reaction can be enhanced and maintained due to the large flux of S. As the travel distance toward the center increases, more S will be consumed, transitioning the kinetics-limited surface reaction to a diffusion-limited process. If the enzymatic reaction is very fast, significant depletion of S can outcompete the confinement-induced enhancement and even suppress the reaction in the inner area of the nanogap. Using a configurable micropost device, the disclosed method can control the mass transport to enable topological modulation of the reaction kinetics and patterning of the reaction products on a surface.

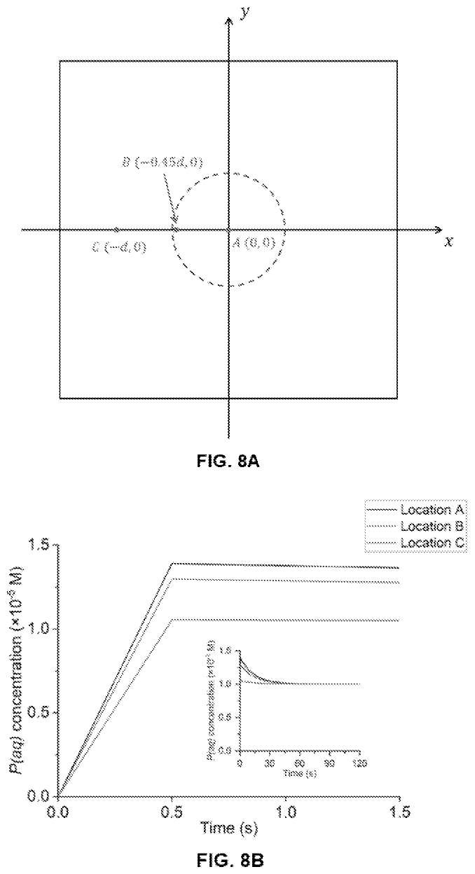

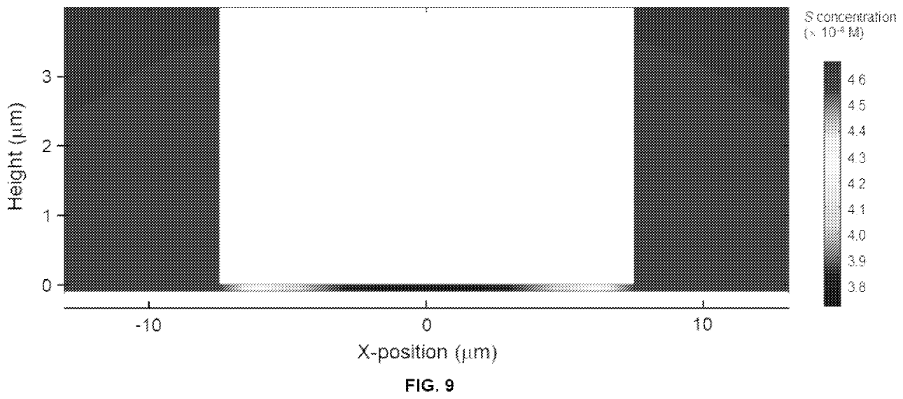

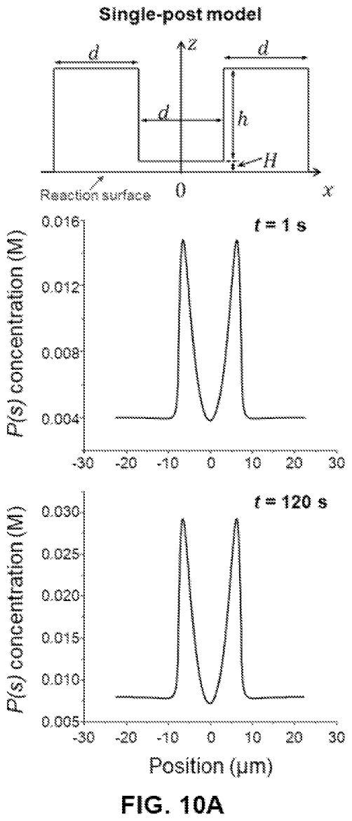

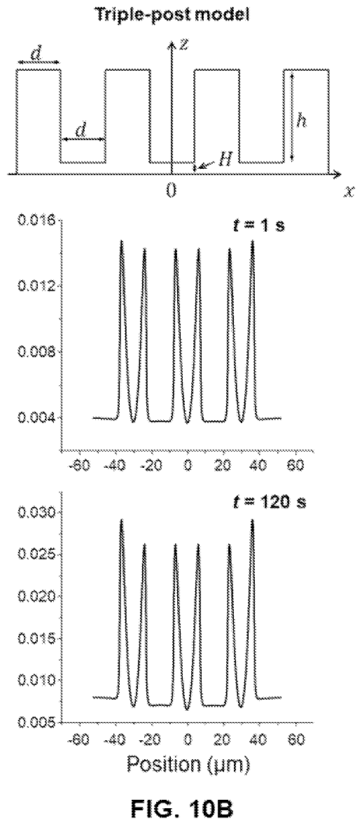

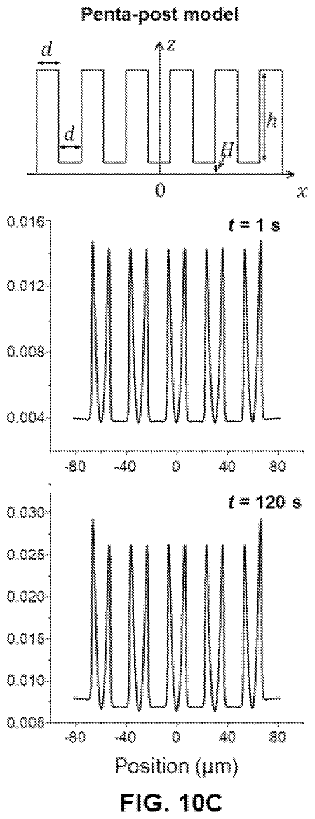

To expedite the computing process, this modeling is focused on the kinetic interplay between mass transport and surface reaction, ignoring other possible molecular-scale factors that may contribute to the enhanced enzyme reactivity under nanoconfinement, including surface charge, conformational change of immobilized proteins, and shift of reaction equilibrium. Given the fact that gap height used here (≥100 nm) is much larger than the calculated Debye length (<1 nm) and the reported dimensions of ALP (~10 nm×5 nm×5 nm for E. coli ALP), such simplification is reasonable and should afford conservative assessment of the surface enzyme kinetics in a multi-length-scale confining system without the need for excessive computational efforts and time. The reaction rates in this model were characterized by a set of first-order rate constants and detailed in Example 4. The time evolution of the surface enzymatic reaction was first simulated in a single-post nanogap system (15 μm in diameter and 100 nm in height, FIGS. 7A-7E). The simulation results show that compared to the open channel surface, the nanogap confinement enhances the reaction rate to reach the saturation level of P(aq) (FIGS. 8A-8B) and significantly elevates the production of P(s) within the nanogap (FIG. 1C). Moreover, the simulated concentration profile of S displays a stronger concentration gradient to drive preferential transport of S from the bulk space to the nanogap versus the open bottom surface (FIG. 9), sustaining the accelerated reaction in the nanoconfinement. Simulation of different multi-post systems yielded the consistent behavior when the ratio of post diameter to post interval was kept the same (FIGS. 10A-10C). These results qualitatively capture the kinetics picture predicted by the model (FIG. 1B) and support the important impacts of micropost confinement on the dynamic interplay between the surface enzyme kinetics and mass transport as described above.

We further assess the nanogap confinement effects by adjusting the simulation variables to probe the processes of surface enzymatic reaction and mass transport. Firstly, the nanogap geometry that directly regulates the mass transport in the disclosed confinement system was adjusted. It is expected that increasing the nanogap radius will restrict mass transport of S to the center and weaken the enhancement effect. Indeed, the simulation with a micropost of 40 μm in diameter showed that the enhancement effect was peaking near the rim of the nanogap and decaying toward the center (FIG. 1D), indicating the transition of the kinetics-limited surface reaction to a diffusion-limited process.

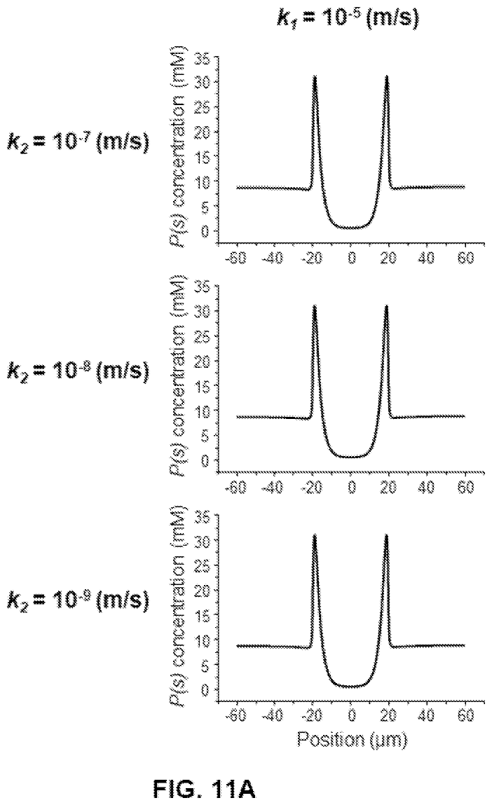

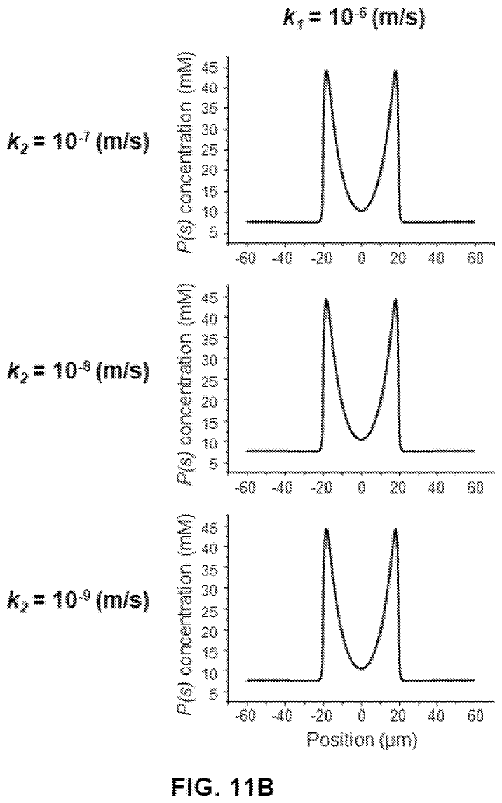

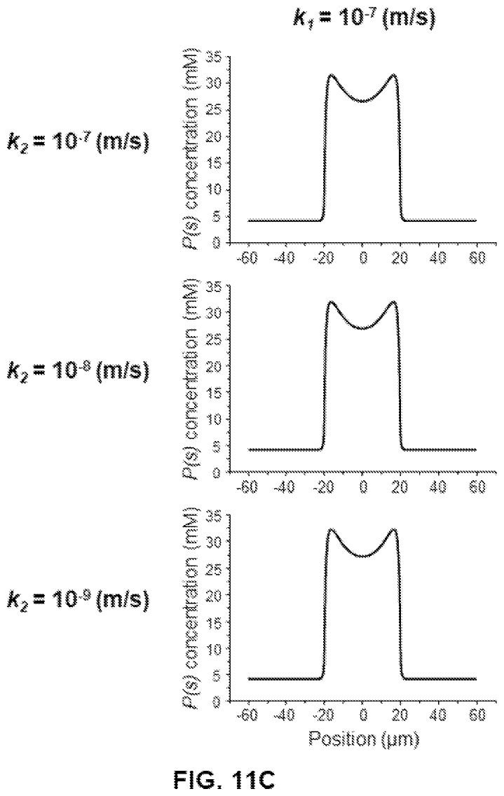

We then tuned the surface reaction kinetics by varying the reaction rate constants. The simulation showed that a relative high reaction rate (e.g., k₁=10⁻⁵ m/s) can lead to reduced enhancement and even notable suppression of the reaction at the central area of the nanogap (FIG. 11A). This can be attributed to the sufficiently fast consumption of S at the open channel surface that depletes the inward supply of S, resulting in a transition from the reaction-limited to diffusion-limited kinetics along the micropost's radius. In contrast, lowering the reaction rate, i.e., reducing k₁ to 10⁻⁶ m/s and 10⁻⁷ m/s, resulted in stronger reaction enhancement at the center of the nanogap (FIGS. 11B-11C), indicating the dominance of diffusive transport of S over the surface consumption of S within the nanogap.

For the 15-μm micropost device, suppression of the reaction enhancement also occurred when increasing the forward reaction rate constant by 10 folds (FIG. 1E). This demonstrates the advantage of smaller confining elements to afford consistent surface reaction enhancement over a broad range of reaction kinetics. The nanogap height is another important dimension in modulating the nanoconfinement effect as it affects mass transport in both radial and vertical direction. The simulation comparing two gap heights of 100 nm and 1 μm formed with a 15-μm micropost was conducted. As seen in FIG. 1F, the nanoconfinement-induced enhancement of the surface enzymatic reaction was almost completely diminished when the gap height was increased to 1 μm. As reasoned previously (FIG. 1B), this result can be attributed to the combination of two effects: 1) reduced enhancement of surface enzyme reactivity under weaker confinement and 2) slower precipitation process because the produced P(aq) near the surface can diffuse away more easily with less vertical spatial restriction. In sum, these results together predict the ability of the micropost-based confining strategy to modulate surface enzymatic reactions via tuning the enzyme kinetics and mass transport.

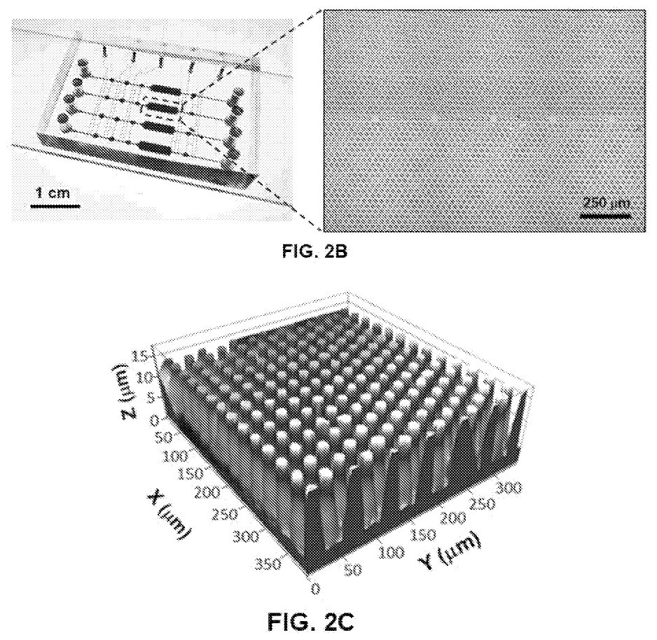

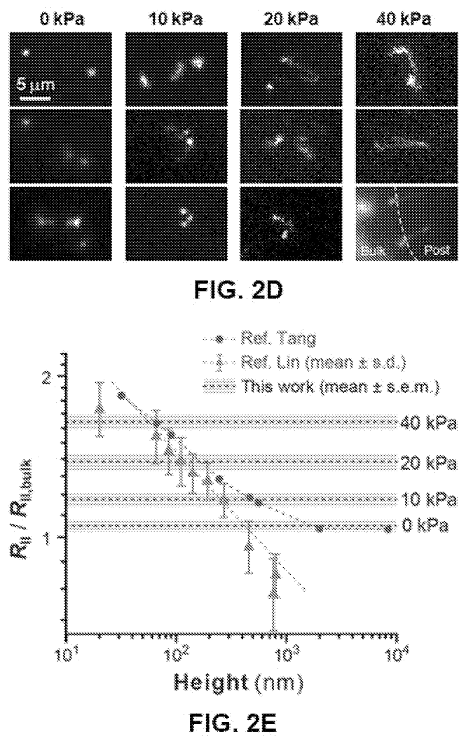

We designed a polydimethylsiloxane (PDMS)/glass hybrid chip composed of a pneumatic control circuit and an array of four parallel microreactors patterned with microposts (FIG. 2A). Each microreactor is flanked by a 3-valve micropump for precise control of reagent delivery and a lifting gate microvalve for stopping the fluid flow for enzymatic reaction. The devices were microfabricated using a multilayer soft lithography process detailed in Example 1. FIG. 2B displays a completed microchip with ~15-μm tall flow channels and the microposts of 15-μm diameter fabricated on a ~150-μm thick PDMS layer. As visualized by the non-contact optical profilometry (FIG. 2C), the fabricated microposts show a conical frustum shape with a slightly reduced top diameter of 11.6±0.9 μm, which is owing to the non-uniform UV exposure across a thick photoresist film resulting in the lithographic structures with non-vertical sidewalls. The diameter and spacing distance of the microposts were varied from 10 to 160 μm, as specified below.

As mentioned previously, the present approach was inspired by the observation that a thin layer of aqueous solution can be formed by pneumatically pressing a PDMS microstructure onto a hydrophilic glass surface under relatively small pressures. Compared to solid thin films, accurate thickness measurement of transparent liquid film at the nanometer scale remains a technical challenge under extensive investigations and mostly relies on optical methods that require highly sophisticated instruments and careful calibration against a reliable reference. A simple method based on fluorescence imaging of spatially confined single DNA molecules provides a more accessible means for convenient estimation of the dimensions of nanofluidic structures. Therefore, this approach was adopted to characterize the slit-like gap created between a micropost and the substrate by visualizing the conformational changes of individual λ-DNA molecules in relation to the characteristic confining dimension which is the gap height (H). The full contour length and the bulk radius of gyration (R_{g,bulk}) of 48.5 kb λ-DNA stained with the YOYO-1 dye in a good solvent have been experimentally measured to be in the range of ~18-25 μm and ~0.7-1 μm, respectively, depending on the measurement methods, dye to base pair ratio, and solvent conditions. FIG. 2D shows typical images of YOYO-1-labeled λ-DNA (dye to base pair ratio of 1:6) confined by the microposts of 80-μm diameter at different pressing pressures. Most of the λ-DNA molecules observed at 0 kPa resembled free-solution DNA in globular random coil conformation with only slight deformation. As the pressing pressure was elevated, λ-DNA became more anisotropically extended; and at 40 kPa linear chains were commonly seen, whose length can reach >50% of the full contour length of λ-DNA (FIG. 2D). Such changes in λ-DNA conformation agree qualitatively with the transition from the weak confinement when H~²R_{g,bulk} to the moderate (Kuhn length L_{K}<H<R_{g,bulk}) and strong confinements (H<L_{K}) that was observed in the fabricated nanoslits with a height ranging from ~30 nm to 2 μm.

For more quantitative assessment of the micropost confinement, the averaged in-plane radius of gyration was measured for λ-DNA floating in the microchannel (R_{∥,bulk}) and confined under the microposts (R∥) as described before (molecule number n>50 for each condition). The measured R_{∥,bulk} (0.80±0.11 μm) for λ-DNA in 1×TE buffer is in line with the values reported with the same labeling ratio and similar TE buffers. FIG. 2E presents the scaled in-plane radius of gyration of λ-DNA (R_{∥}/R_{∥, bulk}) determined at different pressures and the reference plots of R_{∥}/R_{∥,bulk} as a function of slit height reported from two independent studies. Considering the observed weak slit confinement of λ-DNA and the reference plot covering a broad range of nanoslit height (~32 nm to 8.5 μm), the gap height created at 0 kPa (H_{0 kPa}) was estimated to be within ~1.3-2 μm. Given the quantitative discrepancy between two reference plots in the moderate confinement regime (de Gennes regime), the experimental estimate of the slit height at 10 kPa falls in a range of H_{10 kPa}=~270-600 nm, which is much larger than the typical Kuhn length for dsDNA (L_{K}=~100 nm). The nanogap confinement formed at 20 and 40 kPa appears to transition into the Odijk regime where the two reference plots agree well, allowing extraction of their height estimates to be H_{20 kPa}=~100-190 nm and H_{40 kPa}=~50-80 nm. These H estimates also agree well with the values extracted from other relevant studies carried out with various experimental and simulation conditions. It is noted that, despite its simplicity and convenience, the DNA imaging method yields semiquantitative measurement of the confining geometry. More accurate and precise characterization of the nanogap formation in the disclosed device requires systematic investigation using the sophisticated optical methods for liquid thin film analysis, which is beyond the scope of this exploratory proof-of-concept study.

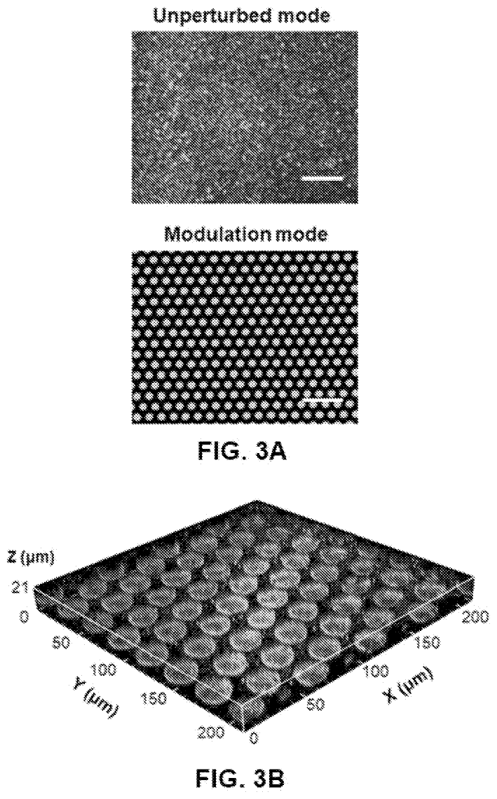

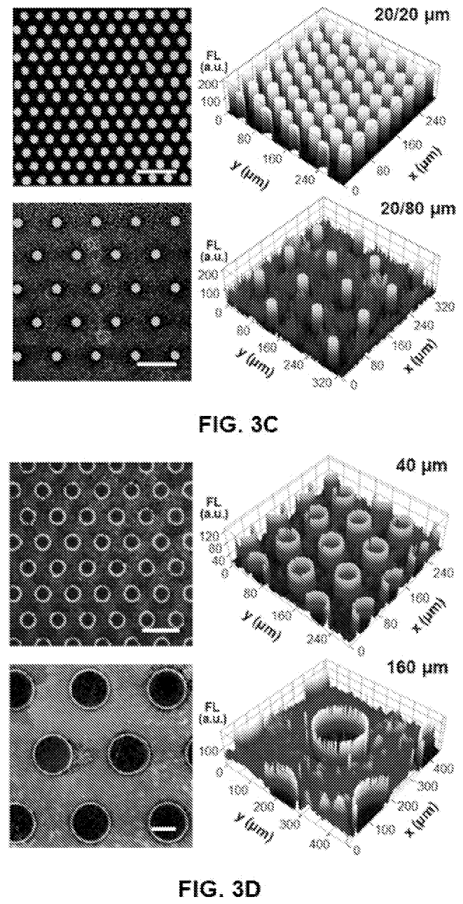

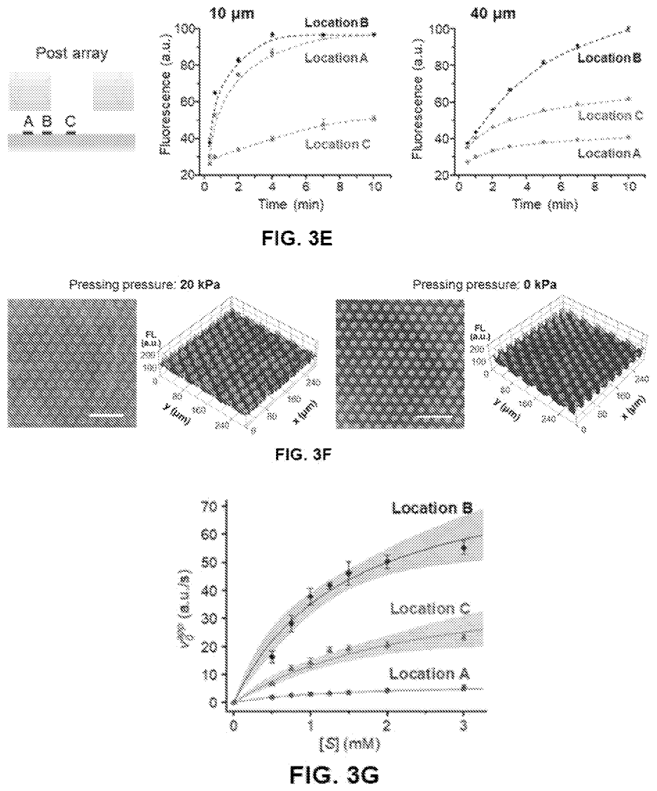

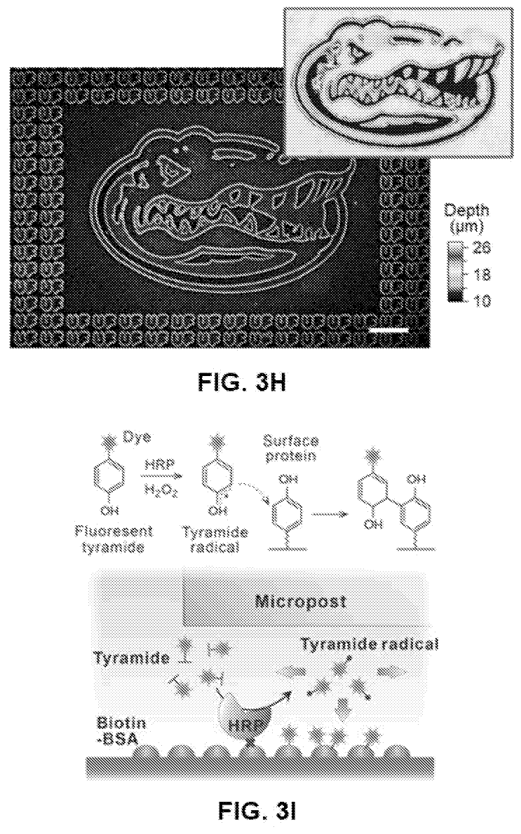

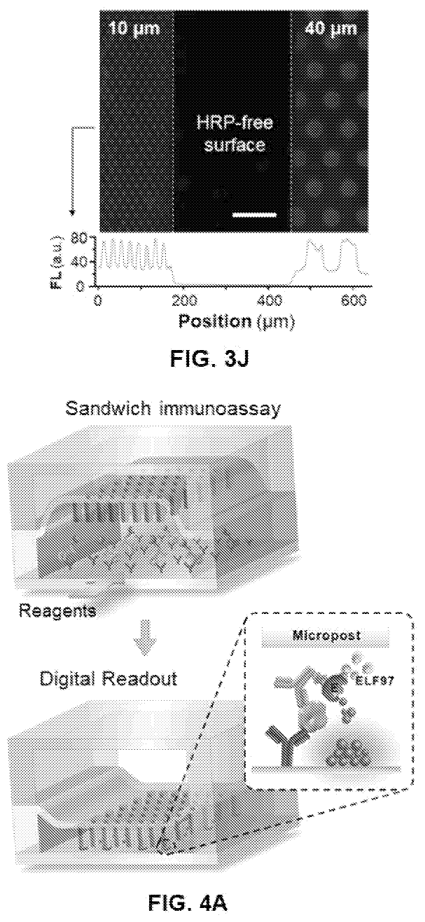

We experimentally assessed the modulation of surface ALP/ELF-97 reaction enabled by the μTUNER strategy (see Example 1). Using an array of posts with the 15-μm diameter and 15-μm spacing, the reaction (1.2 μg/mL streptavidin conjugated ALP and 0.5 mM ELF-97) conducted with the post array lifted (the unperturbed mode) or pressed down (the modulation mode) was first compared, as illustrated in FIGS. 1Ai-1Aiv. As expected, the unperturbed enzymatic reaction led to uniform surface distribution of the fluorescent ELF-97 alcohol precipitates. On the contrary, with the microposts pressed down, the enzymatic production of the precipitates was greatly enhanced under the entire confined regions, while the reaction on the unmasked surface was suppressed (FIG. 3A). Using confocal fluorescence microscopy, it was observed that the ELF-97 alcohol precipitates were mostly generated underneath the microposts, especially near the edge of microposts, rather than the open channel surface or the side walls of the microposts (FIG. 3B). This observation agrees qualitatively with the behavior of the nanogap-modulated surface reaction predicted by this model (FIG. 1C), which confirms the micropost-defined nanoconfinement as the dominant factor to induce the non-contact patterning of surface reaction.

We further investigated these nanoconfinement effects under the same assay conditions via varying the geometrical design of the micropost array. The microposts with a diameter increased to 20 μm could also effectively enhance the ALP/ELF-97 reaction across the confined area underneath (FIG. 3C). Same as seen in FIG. 3A, the reaction on the open channel surface was largely suppressed in the micropost array with the 20-μm spacing (FIG. 3C, top), indicating the overlapped depletion zones formed around microposts due to preferential transport of the substrate to the confined regions (FIG. 9). This was verified by the observation of the separate depletion zones around individual microposts when the spacing was increased to 80 μm (FIG. 3C, bottom). When the post diameter was increased to 40 μm or larger with the same diameter/spacing ratio, a donut-shaped distribution of the fluorescent product was displayed, indicating the intensified enzymatic reaction around the rim of the nanogap and the suppressed reaction in the middle area (FIG. 3D). The micropost-confined reaction kinetics were quantitatively characterized by monitoring the real-time fluorescence signals at three surface locations in a micropost array: (A) the center of a post, (B) the inner point that is ~2 μm from the post edge, and (C) the middle point between two adjacent posts (FIG. 3E, left). For an array of 10-μm posts, the average signals measured at the locations A and B increase at a rate enhanced by ~2.4 folds and ~2.2 folds of that at the location C, respectively, over the first 2 min reaction time (FIG. 3E, middle). While increasing at a slightly lower rate, the signal levels at the center location A eventually approached that at the location B, indicating the confined reaction being dominantly governed by the surface reaction kinetics rather than the diffusive transport of the substrate and reaction products. On the contrary, for an array of 40-μm posts, the signal level at the location A was reduced drastically and lower than that at the location C (FIG. 3E, right). Such post size-dependent change in the surface reaction landscape matches nicely with the simulation results (FIGS. 1C-1D), which manifests the spatial transition from the reaction-limited to the mass transport-limited enzymatic kinetics inward along the radius of a nanogap.

We then investigated the effects of reaction kinetics and mass transport on the nano-confined surface reaction by adjusting the experimental variables that govern the reaction rate and spatial confinement, respectively. As depicted in FIG. 3F (left), when the ELF-97 concentration was increased from 0.5 to 2.5 mM, the donut-shaped patterning of the surface ALP/ELF-97 reaction could also be obtained with smaller microposts, such as the 15-μm microposts. This observation is in line with that of the simulation studies (FIGS. 1E, 11A-11C) where the surface reaction was expedited to suppress the diffusive transport of the substrate towards and thus the reaction in the central area of the nanogaps. Enlarging the height of nanogap can promote the mass transfer to enhance the reaction in the center of nanogaps. As expected, when the 15-μm microposts were raised from <200 nm to ~1-2 μm in height by reducing the pressing pressure from 20 kPa to 0 kPa (FIG. 2E), more uniform enhancement of the reaction across the nanogaps was achieved even at the fast reaction rate (FIG. 3F, right). However, the weaker confinement generated with the lower pressing pressure resulted in less local enhancement of the surface enzymatic reaction, consistent with the theoretical prediction on the effect of nanogap height on the μTUNER process (FIG. 1F).

The nanoconfinement-modulated ALP/ELF-97 reaction kinetics was systematically evaluated with the Michaelis-Menten model. In this case, the enzymatic assays were conducted with an array of 40-μm microposts for which 1.2 μg/mL streptavidin conjugated ALP was used for surface coating and the ELF-97 concentration was varied from 0.5 to 3 mM. The formation of fluorescent precipitates was measured for 10 min at the above-mentioned three surface locations (see Example 1). The apparent initial rates,

$${v}_{0}^{app} ,$$

were plotted against the substrate concentration, [S], and the apparent Michaelis-Menten parameters,

$${K}_{M}^{app} and {V}_{max}^{app} ,$$

were obtained from the fitting curves, as shown in FIG. 3G and Table 4. The key steady-state assumption

$$( \frac{d [ ES ]}{dt} \approx 0 )$$

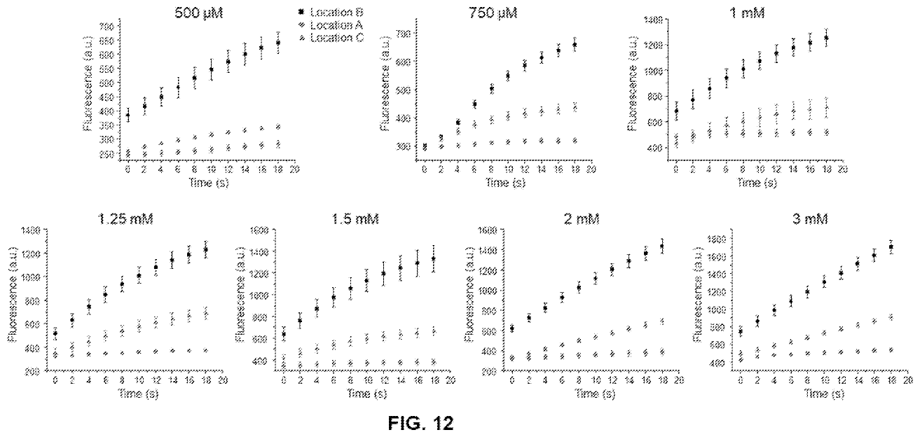

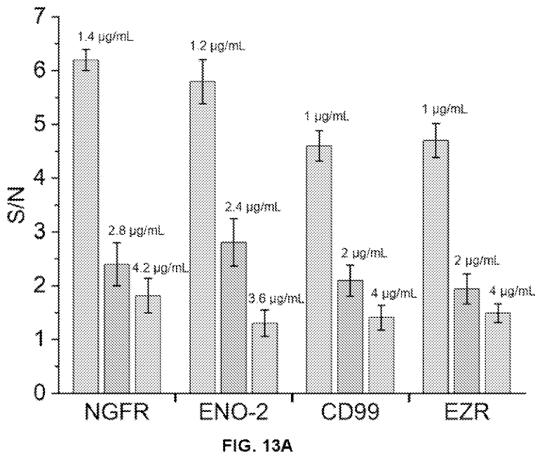

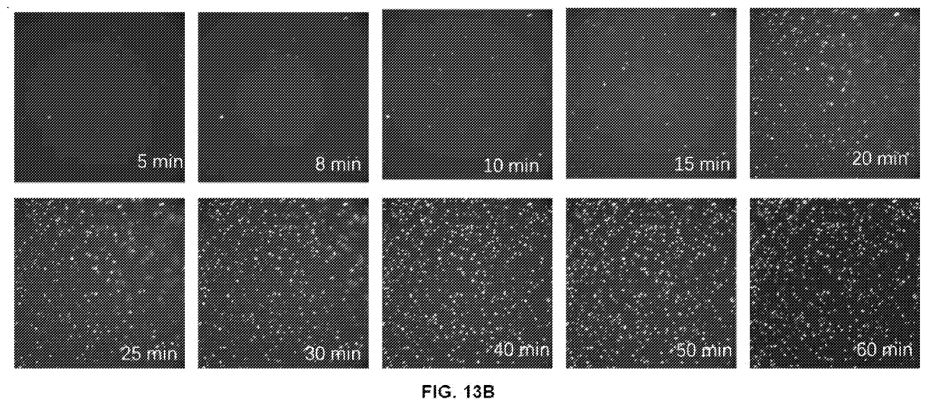

for Michaelis-Menten model holds validity in this study given the linearity of the rate of formation of fluorescent precipitates with respect to the time window of the measurement (FIG. 12). Enzymes exhibited decreasing

$${K}_{M}^{app}$$

from location C to location A, indicating an increasing affinity to the substrates from the open bottom surface to the nanogap center. The improvement of enzyme-substrate affinity can be attributed to the restricted diffusion of the pre-existed substrates within the confined space which allows more interactions with the enzymes relative to the open channel surface. The slightly larger

$${K}_{M}^{app}$$

at location B than at location A can further verify this point as there is a lower spatial impedance on diffusion at the sites near the micropost edge compared to the center of the nanogap. In the meantime, enzymes at location B showed the largest

$${V}_{max}^{app}$$

which is ~12.7 folds and ~1.8 folds of that at locations A and C, respectively.

$${V}_{max}^{app}$$

comprehensively describes the formation of fluorescent precipitates including the conversion of substrates into dissolved products, the precipitation of dissolved products, and the mass transfer of all reaction species. The significant increase in

$${V}_{max}^{app}$$

at location B manifests the nanoconfinement effects to expedite the enzymatic reaction, burst the precipitation of ELF-97 alcohol, as well as sustain the fast reaction via the preferential diffusive transport of substrates into the nanogap. Meanwhile, the magnificent decrease of

$${V}_{max}^{app}$$

at location A suggests a reduced enzymatic reactivity at the nanogap center due to the mass transport-limited kinetics. By further comparing the value of

$$\frac{{V}_{max}^{app}}{{K}_{M}^{app}}$$

which indicates the overall enzymatic efficiency at these three locations, it was discovered that the present approach brings up the best enzymatic performance near the edge of microposts (location B), followed by the open channel surface (location C) and the nanogap center (location A). The results agree well with the prediction of the mechanisms underlying the nanoconfinement effects on surface enzymatic reactions (FIG. 1B) and demonstrate the capability of the present approach to regulate surface enzymatic reactivity and modulate the reactions.

TABLE 4
Apparent Michaelis-Menten Parameters for the Enzymatic Studies

| | Location A | Location B | Location C |
|---|---|---|---|
| ${V}_{max}^{app} ( a . u / s )$ | 6.85 ± 1.34 | 86.74 ± 7.44 | 46.98 ± 5.02 |
| ${K}_{M}^{app} ( mM )$ | 1.31 ± 0.48 | 1.47 ± 0.25 | 2.60 ± 0.42 |

Overall, these experimental findings verify the micropost modulation of the surface enzymatic reaction which enhances the reaction kinetics, promotes the mass transfer of substrate to the nanogap areas, and thus depletes the substrate supply to the unconfined surface reaction. To further explore the capacity of the μTUNER method, the high-resolution, contactless printing of complex patterns on glass substrates was demonstrated by modulating the ALP/ELF-97 reaction. FIG. 3H with a microfabricated positive stamp the University of Florida (UF) hallmarks and Florida Gators. The contour of UF hallmarks and Florida Gators were highlighted by the enhanced production of ELF-97 alcohol precipitates. Collectively, the present studies suggest that the μTUNER strategy affords flexible configurability to modulate the surface enzymatic reaction simply by tunning the nanogap geometry.

As demonstrated above, the μTUNER strategy affords a simple and configurable mechanical approach to engineer biochemical reactions. In addition to the ALP/ELF-97 reaction, the μTUNER strategy was also adapted to modulate the horseradish peroxidase (HRP)/tyramide reaction, which is known as tyramide signal amplification (TSA) or catalyzed reporter deposition (CARD) (FIG. 3I). Different from the heterogeneous process which involves the precipitation of fluorescent enzymatic products, HRP catalyzes the formation of the fluorescent dye-labeled tyramide radicals in the presence of hydrogen peroxide. The short-lived radicals will form covalent bonds with phenol residues on nearby proteins, depositing the fluorescent dye at the site of enzymatic generation. TSA also presents a well-poised signal detection modality as it permits high-density in situ labeling and sensitive visualization of enzymatic activity landscapes as the extremely short lifespan of tyramide radicals limits their diffusion distance upon generation to tens of nm⁹⁶⁻⁹⁸. Here, a mixture array of 40- and 10-μm posts was used for the enzymatic assays for which 2 μg/mL streptavidin conjugated HRP was applied for surface coating followed by reaction with 10× fluorescent dye-labeled tyramide (see Example 1). As shown in FIG. 3J, the fluorescent dye-labeled tyramide formed clear boundaries between the HRP-coated and HRP-free surface region. It was observed that the enhancement of fluorescence signals under both 40- and 10-μm posts compared with those at the unconfined regions. Such an enhancement can be attributed to similar mechanisms underlying the enhancement of ALP/ELF-97 reaction, but specifically for this case, the nanoconfinement enables more tyramide radicals to deposit within their lifespan via restricting the vertical diffusion of tyramide radicals and providing more binding sites compared with the unconfined area. These results demonstrate the potential of the present approach as a more universal strategy to modulate and enhance surface biochemical reactions via simple and configurable mechanical designs.

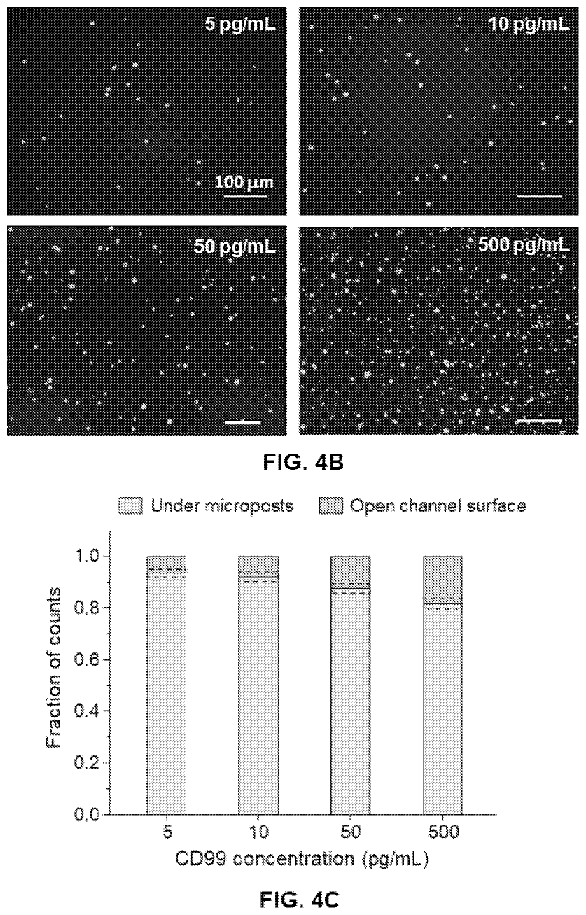

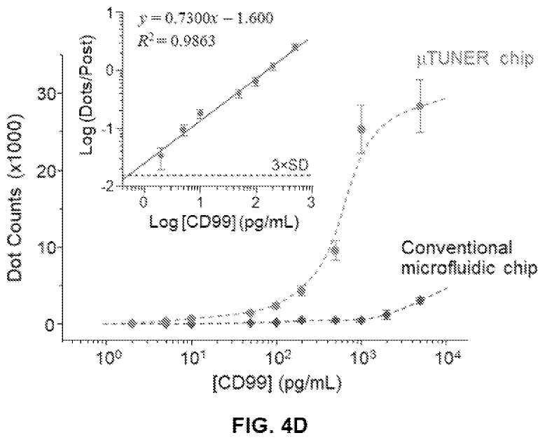

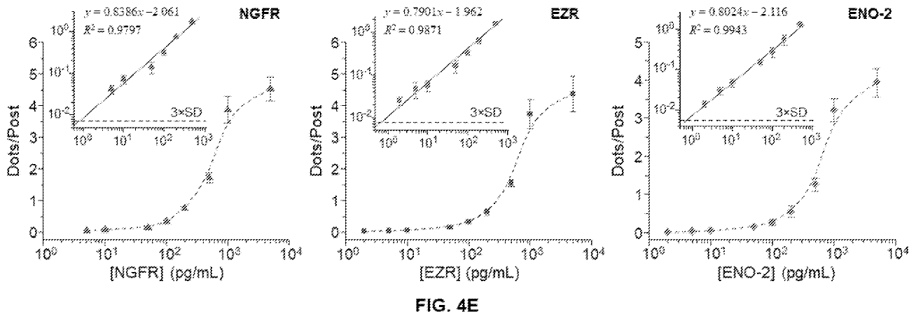

In addition to non-contact surface patterning, the applications of the μTUNER strategy to biosensing were further explored. Inspired by its prominent enhancement effect on the slow reaction kinetics (FIG. 3G), the μTUNER principle was exploited to develop a dELISA system that enables quantitative single-molecule detection of low-level protein targets without the need for sophisticatedly fabricated micro/nanoscale compartments. In this case, the capture antibodies were uniformly immobilized on the glass substrate surface inside the assay chambers (FIG. 2A) using a patterning chip. With the micropost arrays pneumatically lifted, the sample and reagents were flowed through the assay chambers for target immunocapture and the formation of sandwiched immunocomplexes (FIG. 4A, top) using the on-chip micropumps. A stop-flow pumping protocol that was previously established was adopted to promote the efficiency of surface binding and minimize the spatial bias of target capture across the assay chamber. The device was then switched to the μTUNER detection mode by pressing the micropost arrays against the substrate (FIG. 4A, bottom).

We experimentally assessed the μTUNER-enabled dELISA for detection of CD99 protein using the chips equipped with the arrays of 15-μm posts (see Example 1). To demonstrate the single-molecule detection, very low analyte concentrations at the pg/mL levels were used to start. Adopting the convection-reaction-diffusion calculations based on the chip geometries and the typical parameters for continuous-flow microfluidic binding (flow rate Q=1 μL/min, diffusivity D=10 μm²/s, and K_{D}=1 nM), the averaged density of surface-captured molecules under microposts was estimated at a concentration of 5 pg/mL CD99 to be on the order of 1 copy/post. As shown in FIG. 4B, the experiments at the target concentrations of 5 and 10 pg/mL detected individual dots of the fluorescent ELF-97 alcohol precipitate that were mostly formed under different microposts at an average density of approximately 0.09 and 0.18 copies/post, respectively. This observation suggests that the fluorescent dots were produced by individual immunocomplexes and counting the fluorescent dots confers digital single-molecule quantification of targets. The lower density of the observed events than the theoretical prediction can be attributed to the suboptimal affinity binding, loss of captured targets during multiple steps for sandwich ELISA, and heterogenous activity of single reporter enzyme molecules. As the analyte concentration was increased to 50 and 500 pg/mL, the surface density of fluorescent dots escalated so that more dots were detected under the same microposts and some dots started to merge, forming large fluorescent aggregates (FIG. 4B). The ratios of fluorescent dots under the microposts and in the open channel area under these concentrations were quantified (FIG. 4C). At 5 and 10 pg/mL of CD99, 93.7±1.4% and 92.2±2.0% of the fluorescent dots were detected under microposts, respectively, verifying the μTUNER-induced enhancement of single-molecule enzymatic reaction. This percentage reduced slightly down to 87.7±1.8% and 81.8±2.1% with the increased concentrations of 50 and 500 pg/mL, respectively, which can be presumably attributed to the increasing level of aggregation observed (FIG. 4B). Overall, such behavior observed with the compartment-free method resembles qualitatively those of the stochastic compartmentalization of in-solution molecules and surface-bound targets by microdroplets or microchambers for digital single-molecule detection.

Based on a previously established microfluidic ELISA protocol, some key variables were further optimized for the digital immunoassay, i.e., detection antibody concentration and enzymatic reaction time for signal readout (FIGS. 13A-13B). With the optimized assay conditions, the μTUNER chip was calibrated for quantitative detection of CD99, which yielded a dynamic range over three orders of magnitude (FIG. 4D) with a theoretical limit of detection (LOD) of 0.43 pg/mL, calculated from the mean counts for the blank plus three standard deviations (3×S.D., FIG. 4D inset). In parallel, the titration assays were repeated in the conventional manner using the same microfluidic chip without the micropost array. This conventional microfluidic ELISA based on ELF-97 signal amplification yielded an LOD of ~1 ng/mL, which is over 1000-fold higher than that of the μTUNER chip. Such comparison demonstrates the advantage of the μTUNER method in enhancing enzymatic reaction to vastly improve the detection sensitivity of digital immunoassays. In addition to CD99, recent proteomic studies of EWS-derived EVs identified other proteins as potential biomarkers for EWS diagnosis, including nerve growth factor receptor (NGFR), Ezrin (EZR), and enolase 2 (ENO-2). The pneumatically automated multi-channel μTUNER chip allowed establishment of a 4-plex digital immunoassay platform for simultaneous detection of CD99 and these three additional protein targets with the sub-pg/mL LODs (NGFR: 0.81 pg/mL; EZR: 0.66 pg/mL; and ENO-2: 0.70 pg/mL), as shown in FIG. 4E.

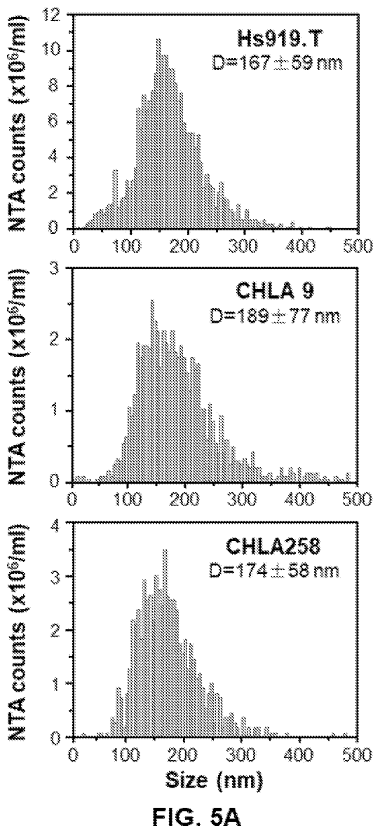

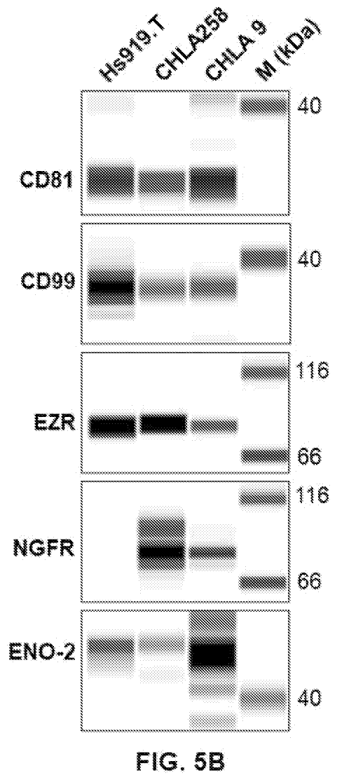

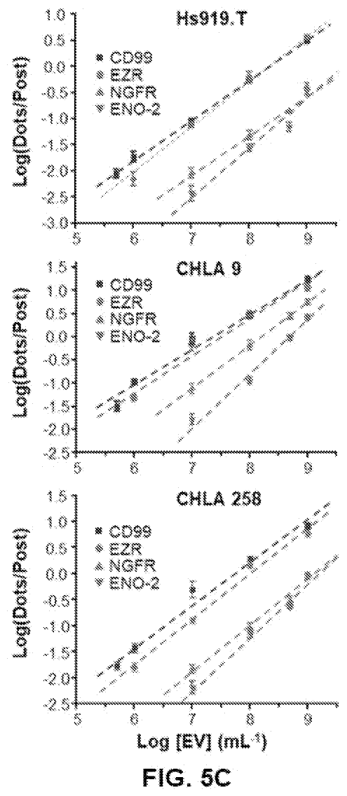

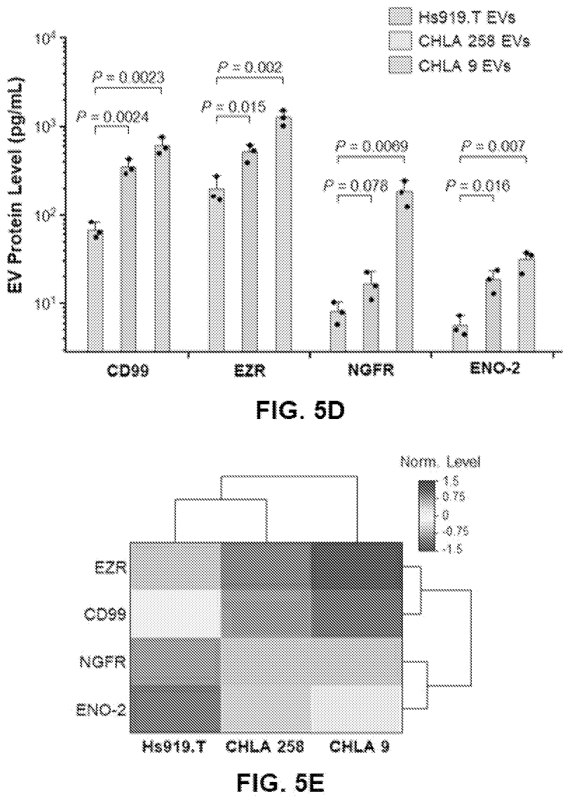

As a proof-of-concept for potential biomedical applications, the μTUNER-enabled digital immunoassay was assessed for EV-based diagnosis of EWS, using cell lines and clinical samples. Small EVs (sEVs) were isolated from both cell culture media and human plasma samples by differential ultracentrifugation (UC) and characterized with the standard molecular assays. FIG. 5A presents the results of nanoparticle tracking analysis (NTA) of EVs isolated from conditioned culture media of a normal control cell line, Hs919.T, and two EWS cell lines, CHLA-9 and CHLA-258. The majority of isolated EVs from the cell lines showed relatively small sizes in a range of ~50-300 nm, which is consistent with previous observations. Using a commercial capillary-based Western analysis system, the expression of four EWS candidate markers (CD99, EZR, NGFR, and ENO-2) and a tetraspanin marker for EVs (CD81) were assessed in these cell line-derived sEVs. As seen in FIG. 5B, CD99, EZR, and ENO-2 were detected in sEVs from all three cell lines, while NGFR was only detectable in two EWS-derived sEVs. These results agree qualitatively with that reported by the sEV proteomic studies, which warrants further quantitative assessment for the application to EWS detection. Isolated sEVs were also quantified by NTA and used for preparing EV standards for quantitative calibration of the μTUNER immunoassays. EV standards were lysed with RIPA lysis buffer and diluted in the assay buffer to minimize the effects of surfactant, followed by injecting the lysate into the microfluidic chip for protein detection (see Example 1). The titration experiments demonstrated excellent performance of the digital immunoassay device for quantitative detection of four proteins in EVs of varying concentrations (FIG. 5C). It is noted that the average level of each marker in sEVs differ across the three cell lines and sEVs from each cell line transport variable quantity of the four markers. Such heterogeneity affects the practical analytical performance for EV markers. For instance, the disclosed method can detect CD99 and Ezrin at a level below 1×10⁶ EVs/mL for these cell lines, as opposed to a higher level of 1×10⁷ EVs/mL required for less abundant NGFR and ENO-2 (FIG. 5C). Moreover, the levels of the markers measured in an equal quantity of isolated sEVs (1×10⁸ EVs/mL) for each cell line were compared. These markers were seen to significantly enriched in the two EWS cell-derived sEVs compared to the normal Hs919.T sEVs, except for NGFR in CHLA258 EVs (FIG. 5D). Combining the expression patterns of four sEV markers defines distinct EV signature for the normal and EWS cell lines, supporting the potential application of EV protein typing for EWS detection (FIG. 5E). Collectively, the cell line studies corroborate that the μTUNER digital immunoassay system affords sensitive and quantitative detection of protein markers in EVs, which warrants subsequent assessment of its adaptability to clinical EWS diagnosis using human specimen.

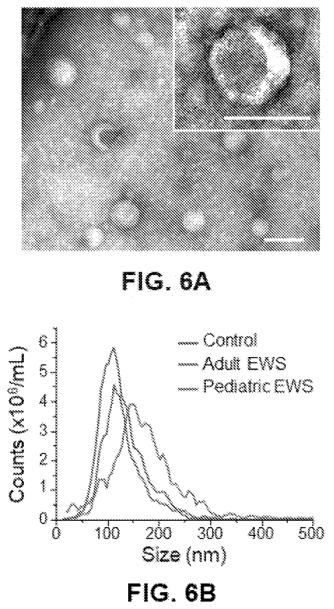

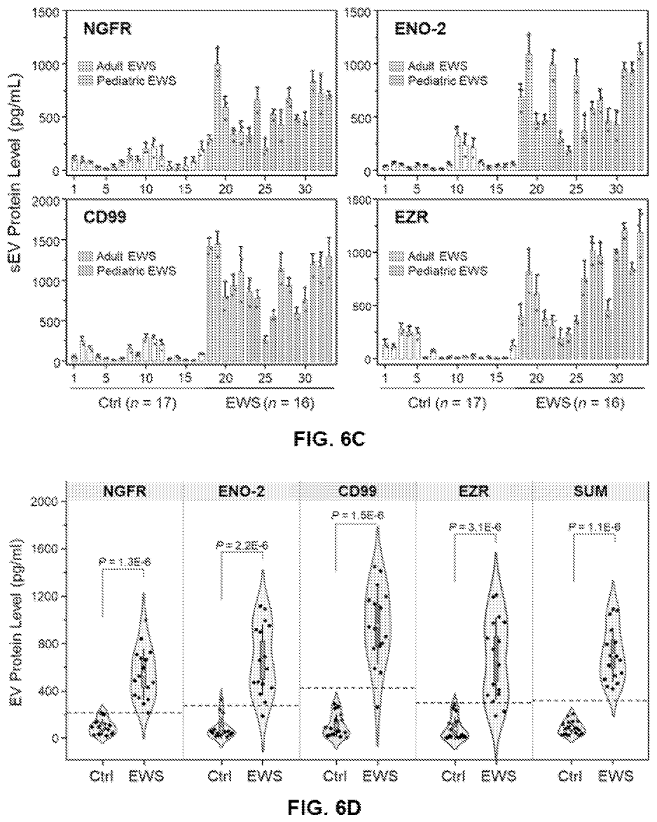

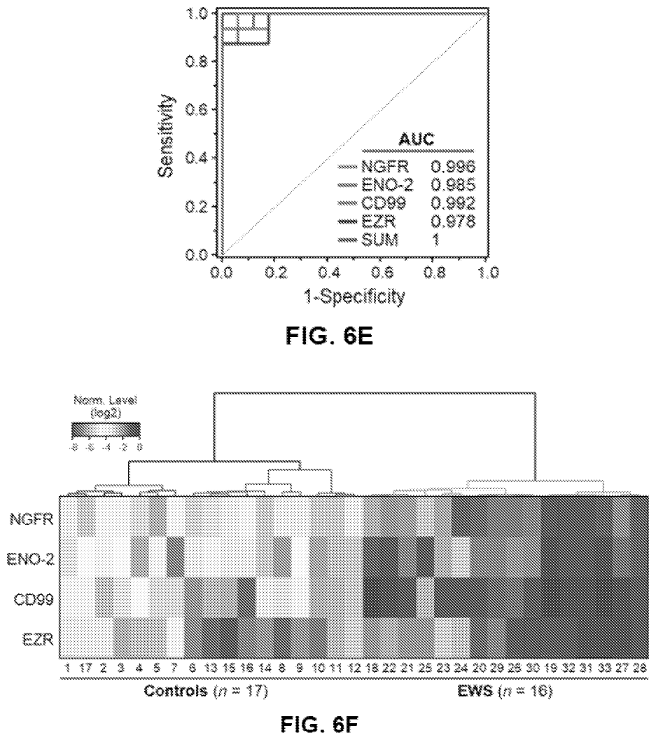

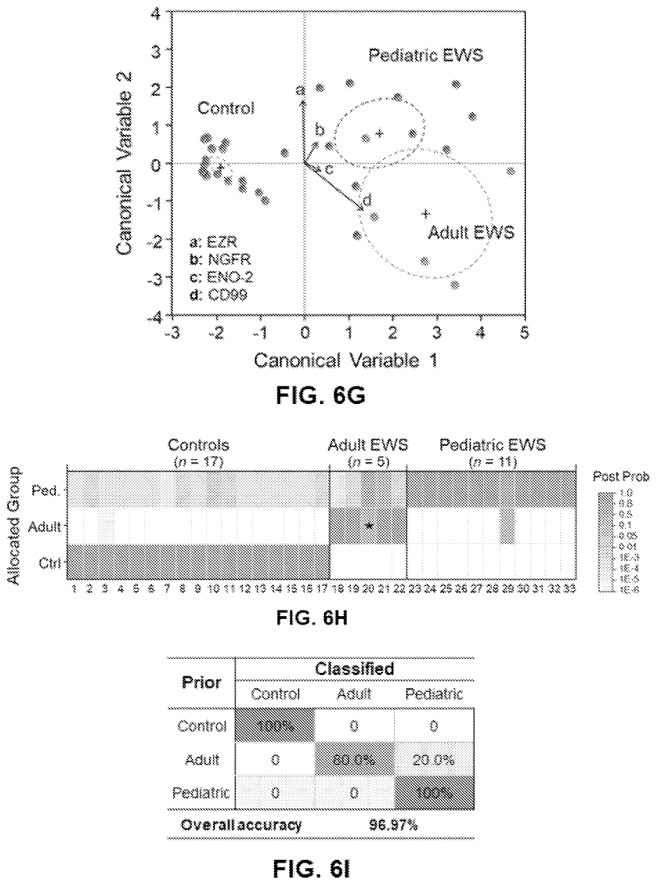

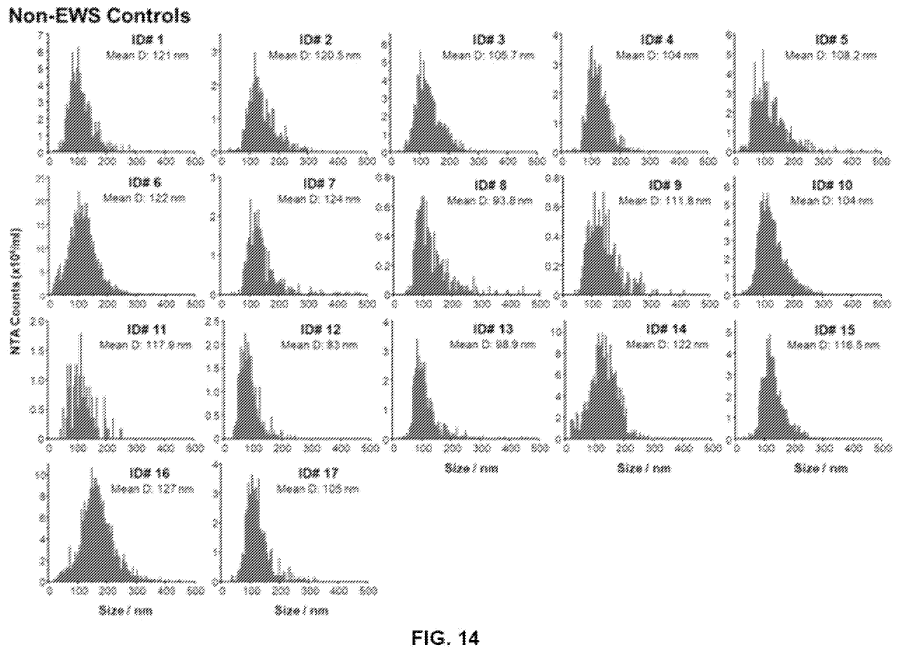

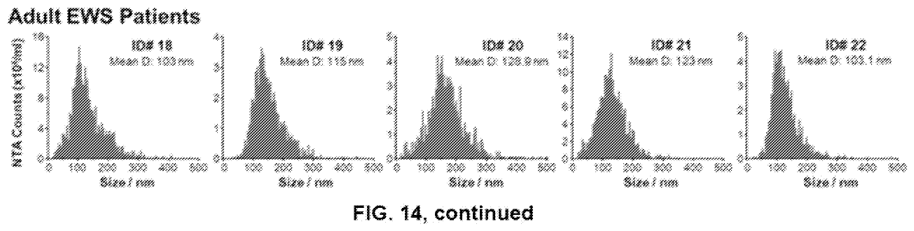

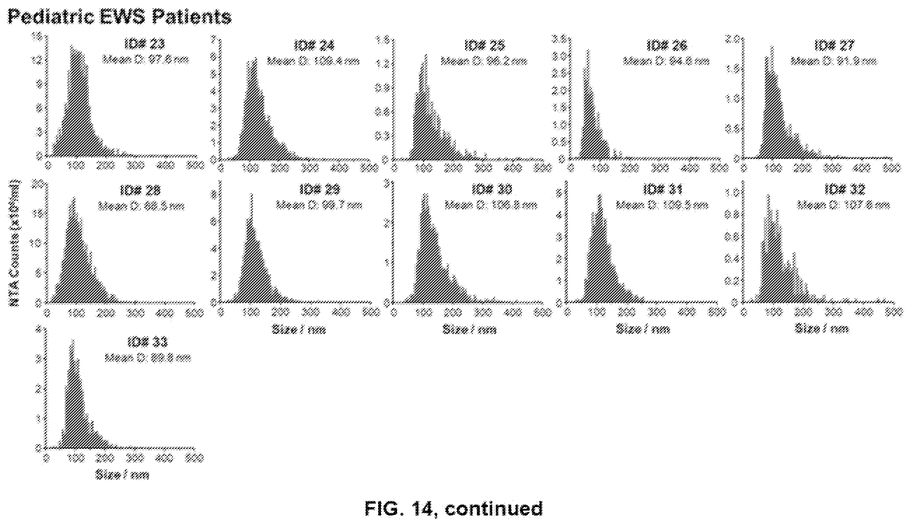

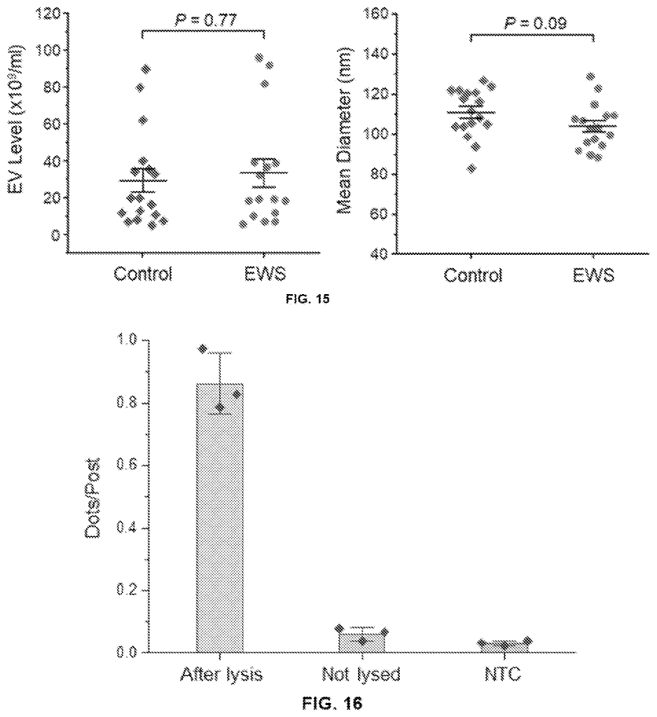

We then assessed the μTUNER-based EV profiling for non-invasive detection of EWS using archival clinical plasma samples representative of a small case-control cohort. EWS primarily occurs in children and young adults (10 to 20 years old) but can also be diagnosed albeit rarely in adults over 40 years old. Therefore, this study was focused on examining an age and gender matched cohort of patients who represent most of this disease population: non-cancer controls (n=17) and EWS patients (n=16) who consist of 11 pediatric (s 18 years of age) and 5 adult patients (19 years old and above), as listed in Table 3. Plasma sEVs were isolated by UC and subjected to quality check using different standard methods. Inspection by transmission electron microscopy (TEM) observed characteristic cup- or round-shaped small vesicles, most of which fall in a size range of ~50-300 nm (FIG. 6A). NTA analysis of the plasma sEV preparations revealed a consistent size range of ~40-300 nm for the majority of EVs and notable subject-to-subject heterogeneity in sEV abundance (5×10⁹ to 9×10¹⁰ EVs/mL) and in size distribution with the mean diameter varying from ~83 to 129 nm (FIGS. 6B, 14). Statistical analysis showed no significant difference in the levels (P=0.77, two-tailed Mann-Whitney U test) and mean sizes (P=0.09) of plasma sEVs between the control and patient groups (FIG. 15). The quality of sEV isolation was further verified by assessing the effect of residual free proteins in the sEV preparation on detection of specific protein markers. To this end, ENO-2 protein, a relatively abundant cytosolic protein, was targeted in sEVs purified from a patient plasma sample using the μTUNER chip. Compared to the blank, no significant digital detection signal for ENO-2 was obtained for assaying the as-prepared sEV sample, while chemical lysis of the same sample resulted in a substantial increase in the target molecules detected, indicating the minuscule effect of residual free proteins on specific biomarker detection (FIG. 16).

Using the multiplexed μTUNER system, the expression of four candidate markers, CD99, EZR, NGFR, and ENO-2 was quantified simultaneously in sEVs derived from each subject. As presented in FIG. 6C, these four candidate markers were found to be elevated in most of the EWS patient-derived sEV samples than the controls, despite considerable interpatient variations observed. Two-sample statistic comparison shows that the control and EWS groups were readily differentiated by the mean levels of individual sEV markers (NGFR: P=1.3E-6; ENO-2: P=2.2E-6; CD99: P=1.5E-6; EZR: P=3.1E-6; two-tailed Mann-Whitney U test) and the discrimination power can be further improved by the unweighted sum of four sEV markers (SUM signature, P=1.1E-6). The diagnostic metrics of the individual biomarkers and SUM signature was quantitatively evaluated using the receiver operating characteristic (ROC) curve and the area under the curve (AUC) analysis (FIG. 6E). It was seen that these sEV markers individually afforded excellent diagnostic performance (AUC values 0.978) to differentiate the EWS and control groups and the SUM signature combining the four proteins further improves the diagnostic power to yield an AUC of 1.00. To demonstrate the feasibility of the digital EV analysis technology for multiplexed disease fingerprinting, a non-supervised hierarchical clustering analysis of the measured levels of four EWS markers was assessed for EWS diagnosis. The resultant heatmap (FIG. 6F) displayed that all subjects were correctly classified into the control and EWS patient groups, respectively, which suggests the clinical potential of the four sEV markers for non-invasive EWS diagnosis and stratification.

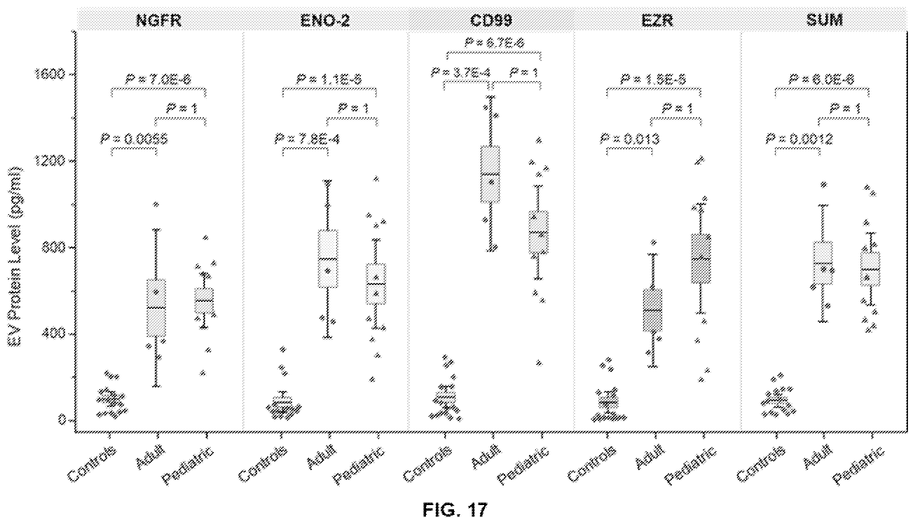

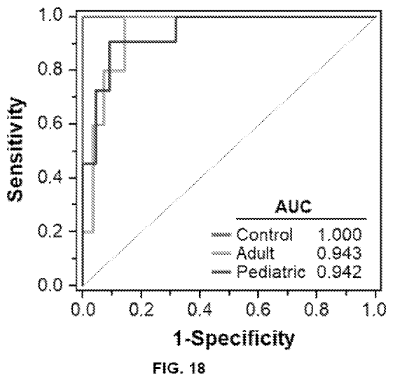

Age has been regarded as one of the clinical prognostic factors that is associated with worse treatment outcomes for young adult patients compared to pediatric patients. Thus, the potential relevance of the present sEV marker-based assay in clinical prognosis of EWS via differentiating the age groups of patients was tested. A statistical comparison of the expression levels of the four sEV markers and the SUM displayed no significant difference between the pediatric and adult groups (FIG. 17). A discriminant analysis of the sEV phenotypes was then tested for subject classification, for which the quadratic mode was chosen as the within-group covariance matrices were tested to be unequal. As visualized in the QDA biplot (FIG. 6G), the measured samples are clustered into three groups: a control group well separated from two distinct groups of the pediatric and adult patients that slightly overlap. The vectors illustrated in this biplot visualize the relative contribution of individual sEV proteins to the separation among the groups, which confirm their diagnostic potential for EWS. Interestingly, the four markers were seen to present two negatively correlated molecular traits in discriminating the EWS patients: EZR and NGFR that classify the pediatric group versus ENO-2 and CD99 for the adult group (FIG. 6G). A quantitative assessment of the QDA classification shows only one misprediction (an adult EWS predicted as pediatric EWS) (FIG. 6H), which yields an overall classification accuracy of 97% (FIG. 6I). The diagnostic performance of the QDA prediction was assessed by the ROC analysis, yielding an AUC value of 1.00, 0.943, and 0.942 for classifying the control, adult, and pediatric group, respectively (FIG. 18). These results suggest the potential adaptation of this technology to evaluate prognostic factors in EWS, in addition to EWS diagnosis as discussed above. Collectively, this proof-of-concept clinical analysis, albeit with the limited sample size, would demonstrate promising clinical value of the disclosed sensitive digital immunoarray device combined with the identified sEV biomarkers as a liquid biopsy-based test, which warrants further validation with larger clinical cohorts.

### Example 3: Discussion

Multi-length-scale engineering has attracted growing interests in biosensing as this strategy marries unique micro- and nano-scale phenomena to immensely improve existing biosensors and develop new sensing mechanisms. In addition to nanomaterials, micro/nanofabricated systems provide a proven platform for constructing precisely defined artificial nanoconfinement devices that enable better control of the fundamental factors governing the reaction equilibrium and kinetics for target binding, amplification, and/or detection. Moreover, these micro/nanochip systems are inherently amenable to the integration of an analytical workflow to build fully integrated biosensing devices. Despite these advantages, there are major challenges in the broad adaptation of these nanodevices to real-world applications. Standard nanofabrication suffers from sophisticated facilities, time-consuming and costly procedures, and limited scalability. Technical challenges can also arise in reproducible operation of nanofabricated devices which requires specialized control instruments and extensive sample processing to mitigate the risk of clogging and surface fouling. Compared to the existing methods, the μTUNER method demonstrated here presents some major advantages: 1) it exploits only simple microfluidic structures to afford configurable formation of nanoscale confinement, substantially promoting the reliability and scalability of device fabrication and operation; 2) this on-demand micro-nanofluidics-convertible mechanism eases direct implementation of various bioassays for analysis of complex samples with minimal pre-treatment; and 3) its inherent compatibility with standard microfluidic engineering could facilitate the development of fully integrated and multiplexed biosensing microsystems. Therefore, the present method could pave a distinct way for developing simple, scalable, and practically viable nanoconfinement technologies to promote their broad applications in basic research and clinical medicine.

Digital analysis has been considered as a transformative paradigm that can lead to radical improvement in the sensitivity, specificity, and accuracy of bioanalysis. Its potential has been well demonstrated by the enormous success of dPCR in the past decade. The prevalent strategy for dPCR relies on the use of physical compartments, such as droplets and microwells, for statistical digitization of target molecules. Recent efforts in translating this concept to digital protein analysis, however, have led to relatively limited advances. This can be attributed in part to the difficulties in fabricating and handling ultrasmall confining structures, e.g., fL-volume microwells, for the detection of single protein molecules that cannot be amplified as in PCR assays. Enabled by the μTUNER method, a compartmentalization-free dELISA system was developed that addresses the major technical difficulties associated with the existing mainstream methods. This method vastly simplifies the device fabrication and the digital assay as it obviates the needs for sophisticated fL-volume devices and/or multi-phase microfluidics for single-molecule partitioning, sealing, and detection. Moreover, the disclosed surface-based dELISA chip further streamlines and expedites the assay workflow by eliminating the multiple steps of off-line immunomagnetic capture and on-chip bead loading. Lastly, in contrast to the array systems with a pre-fixed number of microwells or droplets, the surface-based chip affords a flexible dynamic range for digital counting as it allows continuous injection of variable sample volumes to obtain the number of events desired for Poisson statistics. Such capability is essential to improving the sensitivity and precision for digital detection of low-level targets by directly addressing the low sampling efficiency issue that causes large Poisson noise and uncertainty of single-molecule counting at low concentration. In addition to standard dELISA, a variety of surface-based confinement-free methods have been demonstrated for single-molecule counting of proteins. Among them, enzymatic amplification is a prevailing mechanism for signal enhancement because of its simplicity and low requirements on sophisticated labeling agents and measurement instruments compared to other methods, such as plasmonic probes. These results demonstrated that the simple μTUNER method is applicable to various enzymatic reactions and thus may provide a broadly adaptable platform to enhance the performance of single-molecule counting.

We sought to assess the μTUNER dELISA system using EWS as a model disease because the biomarker-driven research in EWS, as in most pediatric malignancies, lags considerably behind that in adult oncology. The standard diagnostic workup for EWS heavily relies on radiographic imaging which has a very limited capacity for early cancer detection and monitoring of recurrence. While molecular tests, such as the ones based on the immunohistochemical markers, e.g., CD99, or the EWS-specific chimeric fusion genes, have considerably augmented the diagnosis accuracy, they require invasive biopsy sampling of active tumor tissues⁶¹ which are highly invasive, costly, and associated with potential long-term risks. Even successfully treated EWS patients are at high risks of relapse and need to be monitored for years with periodical imaging examinations, which are ineffective for detecting early recurrence and may cause excessive radiation exposure. EWS treatment and monitoring will greatly benefit from non-invasive liquid biopsy diagnostics, which promises to improve early diagnosis and disease monitoring in real-time while avoiding the risks and costs associated with repeated imaging and sedation and invasive biopsies in children. The fields of classic liquid biopsies, e.g., circulating tumor cells (CTCs) and circulating tumor DNA (ctDNA), have witnessed exciting progress in the past decade. Their clinical implementation, however, has been restricted by some serious challenges. Due to the ultralow abundance and heterogeneity of CTCs, reliable isolation and analysis of CTCs are challenging and require a large volume of blood. Analysis of ctDNA can be severely confounded by the vast background of circulating DNA primarily derived from normal cells. Currently, these two liquid biopsy modalities provide limited clinical value depending on clinical applications and cancer types. For instance, a recent study relevant to this work reported the detection of CD99+CTCs in only 4 of 23 patients of EWS in the age range of 13-32 years using 10-mL blood samples. Another next-generation sequencing study detected ctDNA in ~53% of newly diagnosed pediatric patients with localized EWS using a median of 2 mL of archival plasma. It is noted that analysis of CTCs and ctDNA demands a large volume of blood, posing a barrier to their clinical translation in pediatric cancer.

We hypothesized that circulating EVs can provide a promising liquid biopsy for diagnosis of EWS, as accumulating evidence suggests clinical implication of proteomic and RNA cargo selectively packaged into EVs. This hypothesis was supported by pilot studies that identified oncogenic EWS-ETS fusion transcripts (e.g., EWS-FL/1), a hallmark of EWS, and a group of potential protein markers in EWS-derived EVs. Despite their high specificity for EWS diagnosis, EWS-ETS fusion mRNAs were found to be present in small EVs at an ultralow level (0.1-10 copy/10⁵ EVs), which may limit the diagnostic sensitivity. The relatively abundant proteins enriched in EVs offer a promising source to explore EV biomarkers for EWS and other pediatric malignancies. However, a roadblock in the translational studies of pediatric cancers arises from their low incidence and the limited amount of blood samples that can be ethically and safely collected. The disclosed digital ELISA technology immediately addresses this challenge by affording sensitive detection with the sub-pg/mL (i.e., femtomolar level) LODs to enable reliable multiplexed EV profiling using only 20 μL of plasma. These results further support the clinical potential of the four top candidate EV proteins (CD99, NGFR, ENO-2, and EZR) identified by prior proteomic studies as non-invasive EWS markers. Based on the above technical background, this proof-of-concept study, albeit with the limited population size, should have suggested the potential adaptability of the disclosed compartment-free dELISA technology combined with the identified sEV markers as a liquid biopsy test for minimally invasive EWS diagnosis, which warrants further validation with larger clinical cohorts. This technology would also provide a useful tool to facilitate translational studies of EV biomarkers associated with a variety of pediatric malignancies, including other protein markers that had been identified for EWS. By incorporating these protein markers with EWS-ETS mRNAs and microRNAs enriched in EWS-derived EVs, it may be possible to reveal a multi-omic EV signature to improve early diagnosis and even longitudinal monitoring of EWS patients during therapy and then off therapy for recurrence of disease. The development of new enabling technologies like the μTUNER dELSIA would also propel the EV research in EWS to help potentially discover new effective therapeutics that could improve the overall survival of children, adolescent and young adult patients with this debilitating and often fatal disease.

### Example 4: Theoretical Model

### Geometry

The geometry of the simulation model of the micropost-induced perturbation of surface enzymatic reaction is shown in FIGS. 7A-7E. A 3D cuboid microreactor with a single cylindrical post is considered (FIG. 7A). The post is represented by the boundaries created by removing a cylinder from the cuboid. The post is at the center of the microreactor and has a height of h and a diameter of d. The microreactor has a depth and width of 3d, and a height of H+h. The distance between the bottom boundary of the post and the bottom boundary of the microreactor is H, which denotes the nanogap height. The cross-sectional plane at y=0 (FIGS. 7C-7D) further demonstrates the model geometry, including the location of the nanogap. This cross-sectional plane is also adopted as the geometry for the 2D simulation model.

### Equations

The ALP/ELF-97 reaction as the model system to study the μTUNER process is described in FIG. 1B.

$$E + S {{⇌}_{{k}_{2}}}^{{k}_{1}} E \cdot S \to P ( aq ) \to P ( s ) .$$

In the simulation, the reaction model is simplified to a first-order reversible enzymatic reaction¹ happening at the bottom boundary (z=0) of the microreactor (S⇄P(aq)) coupled to a first-order irreversible precipitation happening in the bulk of the microreactor (P(aq)→P(s)):

$$S {{⇌}_{{k}_{2}}}^{{k}_{1}} P ( aq ) {\to }^{{k}_{p}} P ( s ) ,$$

where k₁ and k₂ are the forward and reverse rate constants of the enzymatic reaction, respectively, and kₚ is the rate constant of the precipitation reaction.

We denote the concentration of S, P(aq), and P(s) as c_{A}, c_{P}_{₁}, and c_{P}_{₂}, respectively. The rate of the enzymatic reaction can then be described by

$${r}_{forward} = {k}_{1} {c}_{A} - {k}_{2} {c}_{{P}_{1}} , \qquad ( 1 )$$

$${r}_{reverse} = - {k}_{1} {c}_{A} + {k}_{2} {c}_{{P}_{1}} . \qquad ( 2 )$$

The precipitation reaction is described by a first-order irreversible kinetics model¹. The reaction rate is proportional to the first-order kinetic reaction rate constant kₚ and the difference between the solute concentration c_{P}_{₁} and its saturation concentration Cₛₐₜ^{2,3}.

$${r}_{precipitation} = {k}_{p} ( {c}_{{P}_{1}} - {c}_{sat} ) . \qquad ( 3 )$$

We introduce a control function:

$$φ ( x ) = \{ \begin{matrix}0 , x < 0 \\ 1 , x > 0\end{matrix} . \qquad ( 4 )$$

Since the precipitation reaction will not happen until the solute concentration c_{P}_{₁} reaches its saturation concentration Cₛₐₜ, the control function is coupled with Eq. (3) to describe the precipitation of P(aq) that happens in the bulk. This yields

$${r}_{precipitation} = {k}_{p} ( {c}_{{P}_{1}} - {c}_{sat} ) \cdot φ ( {c}_{{P}_{1}} - {c}_{sat} ) . \qquad ( 5 )$$

Since the fluid flow in the microreactor is stopped to prevent hydrodynamic disturbance of the spatial distribution of the enzymatic reaction products in the experimental design, convection is absent in all transport equations. The three species S, P(aq), and P(s) have bulk diffusivity denoted by D_{A}, D_{P}_{₁}, and D_{P}_{₂}. All equations below are in dimensional form. The diffusion-reaction equations are constructed as follows. Simulation constants can be found in Table 2.

For S, the governing transport equation in the bulk is

$$\frac{\partial {c}_{A}}{\partial t} = {D}_{A} {∇}^{2} {c}_{A} . \qquad ( 6 )$$

The boundary condition for S at the reaction surface (z=0) couples the enzymatic reaction rate:

$$n \cdot ( {D}_{A} ∇ {c}_{A} ) = - {k}_{1} {c}_{A} + {k}_{2} {c}_{{P}_{1}} . \qquad ( 7 )$$

For all other boundaries, the boundary condition is no flux:

$$n \cdot ( {D}_{A} ∇ {c}_{A} ) = 0. \qquad ( 8 )$$

S has an initial concentration of c_{A_0} in the bulk:

$${c}_{A} = {c}_{A \_ 0} . \qquad ( 9 )$$

For P(aq), the governing transport equation in the bulk is

$$\frac{\partial {c}_{{P}_{1}}}{\partial t} = {D}_{{P}_{1}} {∇}^{2} {c}_{{P}_{1}} - {k}_{p} ( {c}_{{P}_{1}} - {c}_{sat} ) \cdot φ ( {c}_{{P}_{1}} - {c}_{sat} ) . \qquad ( 10 )$$

The boundary condition for P(aq) at the reaction surface (z=0) couples the enzymatic reaction rate:

$$n \cdot ( {D}_{{P}_{1}} ∇ {c}_{{P}_{1}} ) = {k}_{1} {c}_{A} - {k}_{2} {c}_{{P}_{1}} . \qquad ( 11 )$$

For all other boundaries, the boundary condition is no flux:

$$n \cdot ( {D}_{{P}_{1}} ∇ {c}_{{P}_{1}} ) = 0. \qquad ( 12 )$$

P(aq) has an initial concentration of c_{P}_{₁}_{_0} in the bulk:

$${c}_{{P}_{1}} = {c}_{{P}_{1 -} 0} . \qquad ( 13 )$$

For P(s), the governing transport equation in the bulk is

$$\frac{\partial {c}_{{P}_{2}}}{\partial t} = {D}_{{P}_{2}} {∇}^{2} {c}_{{P}_{2}} + {k}_{p} ( {c}_{{P}_{1}} - {c}_{sat} ) \cdot φ ( {c}_{{P}_{1}} - {c}_{sat} ) . \qquad ( 14 )$$

The boundary condition for P(s) at all boundaries is no flux:

$$n \cdot ( {D}_{{P}_{2}} ∇ {c}_{{P}_{2}} ) = 0. \qquad ( 15 )$$

P(s) has an initial concentration of C_{P}_{₂}_{_0} in the bulk:

$${c}_{{P}_{2}} = {c}_{{P}_{2 -} 0} . \qquad ( 16 )$$

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Aizenberg, J., et al. Calcitic microlenses as part of the photoreceptor system in brittlestars. Nature 412, 819-822 (2001).
2. Akama, K., et al. Droplet-free digital enzyme-linked immunosorbent assay based on a tyramide signal amplification system. Anal. Chem. 88, 7123-7129 (2016).
3. Arndt, C. A. S., et al. Common musculoskeletal tumors of childhood and adolescence. Mayo Clin Proc 87, 475-487 (2012).
4. Arvand, A. et al. Biology of EWS/ETS fusions in Ewing's family tumors. Oncogene 20, 5747-5754 (2001).
5. Balamuth, N. J. et al. Ewing's sarcoma. The Lancet Oncology 11, 184-192 (2010).
6. Basu, S. et al. Enzymatic activity of alkaline phosphatase inside protein and polymer structures fabricated via multiphoton excitation. Biomacromolecules 5, 572-579 (2004).
7. Bell, A. T. The impact of nanoscience on heterogeneous catalysis. Science 299, 1688-1691 (2003).
8. Benini, S. et al. Detection of circulating tumor cells in liquid biopsy from Ewing sarcoma patients. CMAR 10, 49-60 (2018).
9. Berg, S. L. Ethical challenges in cancer research in children. The Oncologist 12, 1336-1343 (2007).
10. Bernstein, M. et al. Ewing's sarcoma family of tumors: current management. The Oncologist 11, 503-519 (2006).
11. Bird, G., et al. Silica transport during steam injection into oil sands: 1. Dissolution and precipitation kinetics of quartz: New results and review of existing data. Chemical Geology 54, 69-80 (1986).
12. Bobrow, M. N., et al Catalyzed reporter deposition, a novel method of signal amplification application to immunoassays. Journal of Immunological Methods 125, 279-285 (1989).
13. Bobrow, M. N., et al. Catalyzed reporter deposition, a novel method of signal amplification: II. Application to membrane immunoassays. Journal of Immunological Methods 137, 103-112 (1991).
14. Bobrow, M. N., et al. The use of catalyzed reporter deposition as a means of signal amplification in a variety of formats. Journal of Immunological Methods 150, 145-149 (1992).
15. Boklan, J. Little patients, losing patience: pediatric cancer drug development. Molecular Cancer Therapeutics 5, 1905-1908 (2006).
16. Bonthuis, D. J., et al. Conformation and dynamics of DNA confined in slitlike nanofluidic channels. Phys. Rev. Lett. 101, 108303 (2008).
17. Cao, L. et al. Advances in digital polymerase chain reaction (dPCR) and its emerging biomedical applications. Biosensors and Bioelectronics 90, 459-474 (2017).
18. Chen, G., et al. Nanochemistry and nanomedicine for nanoparticle-based diagnostics and therapy. Chem. Rev. 116, 2826-2885 (2016).
19. Cheng, S. et al. Advances in microfluidic extracellular vesicle analysis for cancer diagnostics. Lab Chip 21, 3219-3243 (2021).
20. Chi, J., et al. Generating microdroplet array on photonic pseudo-paper for absolute quantification of nucleic acids. ACS Appl. Mater. Interfaces 10, 39144-39150 (2018).
21. Cohen, L. et al. Single-molecule arrays for protein and nucleic acid analysis. Annual Review of Analytical Chemistry 10, 345-363 (2017).
22. Cox, W. G. et al. A high-resolution, fluorescence-based method for localization of endogenous alkaline phosphatase activity. J Histochem Cytochem. 47, 1443-1455 (1999).
23. Crow, J. et al. MicroRNA content of Ewing sarcoma derived extracellular vesicles leads to biomarker potential and identification of a previously undocumented EWS-FLI1 translocation. Biomark Insights 17, 11772719221132692 (2022).
24. Dai, L., et al. A systematic study of DNA conformation in slitlike confinement. Soft Matter 8, 2972-2982 (2012).
25. Deen, W. M. Analysis of Transport Phenomena. (Oxford University Press, 2012).
26. Dhanjai, et al Robust single-molecule enzyme nanocapsules for biosensing with significantly improved biosensor stability. Anal. Chem. 92, 5830-5837 (2020).
27. Dijk, P. et al. Precipitation and dissolution of reactive solute in fracture. Water Resources Research—WATER RESOUR RES 34, 457-470 (1998).
28. Dong, B. et al. Deciphering nanoconfinement effects on molecular orientation and reaction intermediate by single molecule imaging. Nat Commun 10, 4815 (2019).
29. Farka, Z. et al. Advances in optical single-molecule detection: en route to supersensitive bioaffinity assays. Angewandte Chemie International Edition 59, 10746-10773 (2020).
30. Ferraro, V., et al. Full-field and quantitative analysis of a thin liquid film at the nanoscale by combining digital holography and white light interferometry. J. Phys. Chem. C 125, 1075-1086 (2021).
31. Galvin, C. J., et al. Total capture, convection-limited nanofluidic immunoassays exhibiting nanoconfinement effects. Anal. Chem. 90, 3211-3219 (2018).
32. Garcia-Martin, R. et al. MicroRNA sequence codes for small extracellular vesicle release and cellular retention. Nature 601, 446-451 (2022).
33. Gilboa, T., et al. Single-molecule studies reveal method for tuning the heterogeneous activity of alkaline phosphatase. Biophysical Journal 121, 2027-2034 (2022).
34. Grodzinsky, A. J. Fields, Forces, and Flows in Biological Systems. (Garland Science, 2011).
35. Grommet, A. B., et al. Chemical reactivity under nanoconfinement. Nat. Nanotechnol. 15, 256-271 (2020).
36. Grzybowski, B. A. et al. The nanotechnology of life-inspired systems. Nature Nanotech 11, 585-592 (2016).
37. Hajian, R. et al. Detection of unamplified target genes via CRISPR-Cas9 immobilized on a graphene field-effect transistor. Nat Biomed Eng 3, 427-437 (2019).
38. Hansma, H. G. The power of crowding for the origins of life. Orig Life Evol Biosph 44, 307-311 (2014).
39. He, M., et al. Integrated immunoisolation and protein analysis of circulating exosomes using microfluidic technology. Lab Chip 14, 3773-3780 (2014).
40. Hindson, B. J. et al. High-throughput droplet digital PCR system for absolute quantitation of DNA copy number. Anal. Chem. 83, 8604-8610 (2011).
41. Holtz, K. M. et al. The mechanism of the alkaline phosphatase reaction: insights from NMR, crystallography and site-specific mutagenesis. FEBS Letters 462, 7-11 (1999).
42. Hooley, R. J. Taking on the turnover challenge. Nature Chem 8, 202-204 (2016).
43. Huang, Z., et al. 2-(2′-Phosphoryloxyphenyl)-4(3H)-quinazolinone derivatives as fluorogenic precipitating substrates of phosphatases. Analytical Biochemistry 207, 32-39 (1992).
44. Hughes, A. J. et al. Single-cell western blotting. Nat Methods 11, 749-755 (2014).
45. Jain, M., et al. The Oxford Nanopore MinION: delivery of nanopore sequencing to the genomics community. Genome Biology 17, 239 (2016).
46. Ji et al. Enhanced protein digestion through the confinement of nanozeolite-assembled microchip reactors. Anal. Chem. 80, 2457-2463 (2008).
47. Jo, K. et al. A single-molecule barcoding system using nanoslits for DNA analysis. Proceedings of the National Academy of Sciences 104, 2673-2678 (2007).
48. Kalluri, R. et al. The biology, function, and biomedical applications of exosomes. Science 367, eaau6977 (2020).
49. Kelley, S. O. et al. Advancing the speed, sensitivity and accuracy of biomolecular detection using multi-length-scale engineering. Nature Nanotech 9, 969-980 (2014).
50. Kim, T.-I., et al A fluorescent turn-on probe for the detection of alkaline phosphatase activity in living cells. Chem. Commun. 47, 9825-9827 (2011).
51. Küchler, A., et al Enzymatic reactions in confined environments. Nature Nanotech 11, 409-420 (2016).
52. Lei, C. et al. Characterization of functionalized nanoporous supports for protein confinement. Nanotechnology 17, 5531 (2006).
53. Lei, C., et al. Entrapping enzyme in a functionalized nanoporous support. J. Am. Chem. Soc. 124, 11242-11243 (2002).
54. Li, S.-J. et al. Real-time monitoring of mass-transport-related enzymatic reaction kinetics in a nanochannel-array reactor. Chemistry—A European Journal 16, 10186-10194 (2010).
55. Li, Y., et al. Wettability-patterned microchip for emerging biomedical materials and technologies. Materials Today 51, 273-293 (2021).
56. Lin, P., et al. Effects of topology and ionic strength on double-stranded DNA confined in nanoslits. Macromolecules 45, 2920-2927 (2012).
57. Liu, J., et al. Molecular-based design and emerging applications of nanoporous carbon spheres. Nature Mater 14, 763-774 (2015).
58. Lynn, N. S., et al. Enhancement of affinity-based biosensors: effect of sensing chamber geometry on sensitivity. Lab Chip 13, 1413-1421 (2013).
59. Maley, A. M., et al. Simplified digital enzyme-linked immunosorbent assay using tyramide signal amplification and fibrin hydrogels. ACS Sens. 5, 3037-3042 (2020).
60. Mann, S. The origins of life: old problems, new chemistries. Angewandte Chemie International Edition 52, 155-162 (2013).
61. Miles, B. N. et al. Single molecule sensing with solid-state nanopores: novel materials, methods, and applications. Chem. Soc. Rev. 42, 15-28 (2012).
62. Neira, H. D. et al. Kinetic analysis of enzymes immobilized in porous film arrays. Anal. Chem. 89, 10311-10320 (2017).
63. Noji, H., et al. Enzyme-based digital bioassay technology—key strategies and future perspectives. Lab Chip 22, 3092-3109 (2022).
64. O'Brien, K., et al. RNA delivery by extracellular vesicles in mammalian cells and its applications. Nat Rev Mol Cell Biol 21, 585-606 (2020).
65. Paragas, V. B., et al The ELF-97 alkaline phosphatase substrate provides a bright, photostable, fluorescent signal amplification method for FISH. J Histochem Cytochem. 45, 345-357 (1997).
66. Park, S., et al. A method for nanofluidic device prototyping using elastomeric collapse. Proceedings of the National Academy of Sciences 106, 15549-15554 (2009).
67. Parsa, H. et al. Effect of volume- and time-based constraints on capture of analytes in microfluidic heterogeneous immunoassays. Lab Chip 8, 2062-2070 (2008).
68. Peluso, P. et al. Optimizing antibody immobilization strategies for the construction of protein microarrays. Analytical Biochemistry 312, 113-124 (2003).
69. Ramachandran, P. A. Advanced Transport Phenomena: Analysis, Modeling and Computation. (Cambridge University Press, 2014).
70. Razzaghi, A., et al. Thickness measurement of transparent liquid films with Paraxial Self-Reference Interferometry. Sci Rep 10, 9240 (2020).
71. Rissin, D. M. et al. Simultaneous detection of single molecules and singulated ensembles of molecules enables immunoassays with broad dynamic range. Anal. Chem. 83, 2279-2285 (2011).
72. Rissin, D. M. et al. Single-molecule enzyme-linked immunosorbent assay detects serum proteins at subfemtomolar concentrations. Nat Biotechnol 28, 595-599 (2010).
73. Rissin, D. M., et al. Distinct and long-lived activity states of single enzyme molecules. J. Am. Chem. Soc. 130, 5349-5353 (2008).
74. Rondelez, Y. et al. Highly coupled ATP synthesis by F1-ATPase single molecules. Nature 433, 773-777 (2005).
75. Samuel, G. et al. Ewing sarcoma family of tumors-derived small extracellular vesicle proteomics identify potential clinical biomarkers. Oncotarget 11, 2995-3012 (2020).
76. Schnepp, R. W., et al. Improving patient outcomes with cancer genomics: unique opportunities and challenges in pediatric oncology. JAMA 314, 881-883 (2015).
77. Schweitzer, B. et al. Immunoassays with rolling circle DNA amplification: A versatile platform for ultrasensitive antigen detection. Proceedings of the National Academy of Sciences 97, 10113-10119 (2000).
78. Shang, Y., et al Integrated microfluidic lectin barcode platform for high-performance focused glycomic profiling. Sci Rep 6, 20297 (2016).
79. Shichida, Y. et al. Evolution of opsins and phototransduction. Philos Trans R Soc Lond B Biol Sci 364, 2881-2895 (2009).
80. Shon, M. J. et al. Mass action at the single-molecule level. J. Am. Chem. Soc. 134, 14618-14623 (2012).
81. Shulman, D. S. et al. Detection of circulating tumour DNA is associated with inferior outcomes in Ewing sarcoma and osteosarcoma: a report from the Children's Oncology Group. Br J Cancer 119, 615-621 (2018).
82. Smith, D. E., et al. Dynamical scaling of DNA diffusion coefficients. Macromolecules 29, 1372-1373 (1996).
83. Sparreboom, W., et al. Principles and applications of nanofluidic transport. Nature Nanotech 4, 713-720 (2009).
84. Squires, T. M., et al. Making it stick: convection, reaction and diffusion in surface-based biosensors. Nat Biotechnol 26, 417-426 (2008).
85. Strychalski, E. A., et al. Quantitative measurements of the size scaling of linear and circular DNA in nanofluidic slitlike confinement. Macromolecules 45, 1602-1611 (2012).
86. Szostak, J. W., et al. Synthesizing life. Nature 409, 387-390 (2001).
87. Takahashi, H. et al. Catalytic activity in organic solvents and stability of immobilized enzymes depend on the pore size and surface characteristics of mesoporous silica. Chem. Mater. 12, 3301-3305 (2000).
88. Tang, J. et al. Revisiting the conformation and dynamics of DNA in slitlike confinement. Macromolecules 43, 7368-7377 (2010).
89. Tripp, B. C., et al. Carbonic anhydrase: new insights for an ancient enzyme*210. Journal of Biological Chemistry 276, 48615-48618 (2001).
90. Urban, P. L. Compartmentalised chemistry: from studies on the origin of life to engineered biochemical systems. New J. Chem. 38, 5135-5141 (2014).
91. Vargas-Lara, F. et al. Dimensional reduction of duplex DNA under confinement to nanofluidic slits. Soft Matter 11, 8273-8284 (2015).
92. Wang, C. et al. Nanoconfinement effects: glucose oxidase reaction kinetics in nanofluidics. ChemPhysChem 13, 762-768 (2012).
93. Wang, H., et al. Carbon-based hybrid nanogels: a synergistic nanoplatform for combined biosensing, bioimaging, and responsive drug delivery. Chem. Soc. Rev. 47, 4198-4232 (2018).
94. Wang, L. et al. Fundamental transport mechanisms, fabrication and potential applications of nanoporous atomically thin membranes. Nature Nanotech 12, 509-522 (2017).
95. Wang, T., et al Ultrasensitive microfluidic solid-phase ELISA using an actuatable microwell-patterned PDMS chip. Lab Chip 13, 4190-4197 (2013).
96. Wang, X., et al. Surface modification of glass/PDMS microfluidic valve assemblies enhances valve electrical resistance. ACS Appl. Mater. Interfaces 11, 34463-34470 (2019).
97. Wang, Y., et al. Digital PCR using micropatterned superporous absorbent array chips. Analyst 141, 3821-3831 (2016).
98. Wang, Y., et al. Nanopore sequencing technology, bioinformatics and applications. Nat Biotechnol 39, 1348-1365 (2021).
99. Weir, G. J. et al. Surface deposition from fluid flow in a porous medium. Transp Porous Med 25, 79-96 (1996).
100. Wilke, C. R. et al. Correlation of diffusion coefficients in dilute solutions. AIChE Journal 1, 264-270 (1955).
101. Womer, R. B. et al. Randomized controlled trial of interval-compressed chemotherapy for the treatment of localized Ewing Sarcoma: A report from the Children's Oncology Group. JCO 30, 4148-4154 (2012).
102. Wu, C., et al. Ultrasensitive detection of attomolar protein concentrations by dropcast single molecule assays. J. Am. Chem. Soc. 142, 12314-12323 (2020).
103. Yamamoto, K., et al. Picoliter enzyme reactor on a nanofluidic device exceeding the bulk reaction rate. Analyst 145, 5801-5807 (2020).
104. Yan, H., et al. Advances in analytical technologies for extracellular vesicles. Anal. Chem. 93, 4739-4774 (2021).
105. Yang, K. S. et al. Extracellular vesicle analysis allows for identification of invasive IPMN. Gastroenterology 160, 1345-1358.e11 (2021).
106. Yang, Y. et al Microfluidic communicating vessel chip for expedited and automated immunomagnetic assays. Lab Chip 18, 3830-3839 (2018).
107. Yelleswarapu, V. et al. Mobile platform for rapid sub-picogram-per-milliliter, multiplexed, digital droplet detection of proteins. Proceedings of the National Academy of Sciences 116, 4489-4495 (2019).
108. Yim, W. et al. Enhanced Photoacoustic Detection of Heparin in Whole Blood via Melanin Nanocapsules Carrying Molecular Agents. ACS Nano 16, 683-693 (2022).
109. Yoshizawa, M., et al. Functional molecular flasks: new properties and reactions within discrete, self-assembled hosts. Angewandte Chemie International Edition 48, 3418-3438 (2009).
110. Yu, R.-J., et al. Confined nanopipette sensing: from single molecules, single nanoparticles, to single cells. Angewandte Chemie International Edition 58, 3706-3714 (2019).
111. Zeng, Y., et al. A. High-performance single cell genetic analysis using microfluidic emulsion generator arrays. Anal. Chem. 82, 3183-3190 (2010).
112. Zhang, J. et al. Nanoconfinement effect for signal amplification in electrochemical analysis and sensing. Small 17, 2101665 (2021).
113. Zhang, P. et al. Plasmonic scattering imaging of single proteins and binding kinetics. Nat Methods 17, 1010-1017 (2020).
114. Zhang, P. et al. Ultrasensitive detection of circulating exosomes with a 3D-nanopatterned microfluidic chip. Nat Biomed Eng 3, 438-451 (2019).
115. Zhang, P. et al. Ultrasensitive quantification of tumor mRNAs in extracellular vesicles with an integrated microfluidic digital analysis chip. Lab Chip 18, 3790-3801 (2018).
116. Zhang, P., et al Molecular assessment of circulating exosomes toward liquid biopsy diagnosis of Ewing sarcoma family of tumors. Transl Res 201, 136-153 (2018).
117. Zhang, P., et al. Multiplexed immunophenotyping of circulating exosomes on nano-engineered ExoProfile chip towards early diagnosis of cancer. Chem. Sci. 10, 5495-5504 (2019).
118. Zhang, P., et al. Ultrasensitive microfluidic analysis of circulating exosomes using a nanostructured graphene oxide/polydopamine coating. Lab on a chip 16, 3033 (2016).
119. Zhang, Y. et al. A review on continuous-flow microfluidic PCR in droplets: Advances, challenges and future. Analytica Chimica Acta 914, 7-16 (2016).
120. Zhang, Y. et al. Design and application of metal organic framework ZIF-90-ZnO—MoS2 nanohybrid for an integrated electrochemical liquid biopsy. Nano Lett. 22, 6833-6840 (2022).
121. Zhang, Y. et al. Digital bioassays: theory, applications, and perspectives. Anal. Chem. 89, 92-101 (2017).
122. Zhao, Y., et al. Metal-organic frameworks as photoluminescent biosensing platforms: mechanisms and applications. Chem. Soc. Rev. 50, 4484-4513 (2021).

1. A system comprising:
(a) a first layer comprising a solid substrate;
(b) a second layer comprising a plurality of microposts;
(c) a third layer; and
(d) a pneumatic actuator;
wherein the plurality of microposts protrude towards the first layer;
wherein the second layer is separated by an air gap from the third layer; and
wherein the pneumatic actuator can be activated to create a nanogap between the first layer and the plurality of microposts and to increase or decrease a height of the nanogap between the first layer and the plurality of microposts by increasing or decreasing a height of the air gap, thereby changing a position of the second layer.

2. The system of claim 1, wherein the solid substrate comprises a glass slide.

3. The system of claim 2, wherein the glass slide has been subjected to a hydrophilic surface treatment.

4. The system of claim 1, wherein the solid substrate is planar.

5. The system of claim 1, wherein the second layer comprises polydimethylsiloxane (PDMS), an addition cure silicone rubber comprising recycled polymers, polymethyl methacrylate (PMMA), or any combination thereof.

6. The system of claim 5, wherein the second layer is made from PDMS.

7. The system of claim 6, wherein the second layer is from about 100 μm thick to about 300 μm thick.

8. The system of claim 7, wherein the second layer is about 150 μm thick.

9. The system of claim 1, wherein the third layer comprises PDMS, an addition cure silicone rubber comprising recycled polymers, polymethyl methacrylate (PMMA), or any combination thereof

10. The system of claim 9, wherein the third layer is made from PDMS.

11. The system of claim 1, wherein the third layer is from about 4 mm thick to about 6 mm thick.

12. The system of claim 11, wherein the third layer is about 5 mm thick.

13. The system of claim 1, wherein the microposts have a base diameter of from about 10 μm to about 200 μm.

14. The system of claim 13, wherein the microposts comprise a conical frustum shape with a top diameter from about 70% to about 100% of the base diameter.

15. The system of claim 1, wherein the microposts have a height of about 15 μm.

16. The system of claim 1, wherein the microposts are spaced from about 10 μm to about 200 μm apart, wherein spacing is measured between an edge of a first micropost and an edge of an adjacent micropost.

17. The system of claim 1 wherein the height of the nanogap can be adjusted between about 50 nm and about 2 μm.

18. The system of claim 1, further comprising at least one micropump and at least one microvalve.

19. The system of claim 18, wherein the micropump comprises a 3-valve micropump or a syringe pump.

20. The system of claim 18, wherein the microvalve comprises a lifting gate microvalve.

21. The system of claim 1, wherein the pneumatic actuator is activated at a pressure of from about 0 kPa to about 40 kPa.

22.-50. (canceled)
